(19) United States
(12) Patent Application Publication
(10) Pub. No.: US 2024/0226482 A9
(43) Pub. Date: Jul. 11, 2024

(54) SYSTEM AND METHOD FOR CONTINUOUS ADJUSTMENT OF PERSONALIZED MASK SHAPE

(71) Applicants: ResMed Inc., San Diego, CA (US); ResMed Pty Ltd., Bella Vista, New South Wales (AU); ResMed Sensor Technologies Limited, Dublin (IE)

(72) Inventors: Gregory Robert PEAKE, San Diego, CA (US); Nathan Zersee LIU, Bella Vista, New South Wales (AU); Sakeena DE SOUZA, Bella Vista, New South Wales (AU); Michael Christopher HOGG, Bella Vista, New South Wales (AU); Redmond SHOULDICE, Dublin (IE)

(21) Appl. No.: 18/548,049

(22) PCT Filed: Feb. 28, 2022
(86) PCT No.: PCT/US2022/018178

§ 371 Date: Aug. 25, 2023

Related U.S. Application Data
(60) Provisional application No. 63/154,223, filed on Feb. 26, 2021.
(60) Provisional application No. 63/168,635, filed on Mar. 31, 2021.

(51) Int. Cl. A61M 16/06; A61M 16/00; G06T 7/593; G06V 40/16

(52) U.S. Cl. CPC A61M 16/0605; A61M 16/024; G06T 7/593; G06V 40/171; A61M 2205/15

(57) ABSTRACT

A system and method to match an interface to the face of a user for respiratory therapy. A facial image of the user is stored. Facial features based on the facial image are determined. A database stores facial profiles based on facial features and a corresponding plurality of interfaces. A database stores operational data of respiratory therapy devices with the plurality of corresponding interfaces. A selection engine is coupled to the databases. The selection engine is operative to select an interface for the user from the plurality of corresponding interfaces based on a desired outcome based on the stored operational data and the facial features. The collected data may also be employed to determine whether the selected interface is correctly fitted to the face of the user.

### PRIORITY CLAIM

The present disclosure claims priority to and the benefit of U.S. Provisional Patent Application Ser. No. 63/154,223 filed Feb. 26, 2021 and U.S. Provisional Patent Application Ser. No. 63/168,635 filed Mar. 31, 2021. The contents of those applications are hereby incorporated by reference in their entirety.

### TECHNICAL FIELD

The present disclosure relates generally to interfaces for respiratory therapy devices, and more specifically to a system to better select a mask based on individual user data.

### BACKGROUND

A range of respiratory disorders exist. Certain disorders may be characterized by particular events, such as apneas, hypopneas, and hyperpneas. Obstructive Sleep Apnea (OSA), a form of Sleep Disordered Breathing (SDB), is characterized by events including occlusion or obstruction of the upper air passage during sleep. It results from a combination of an abnormally small upper airway and the normal loss of muscle tone in the region of the tongue, soft palate and posterior oropharyngeal wall during sleep. The condition causes the affected patient to stop breathing for periods typically of 30 to 120 seconds in duration, sometimes 200 to 300 times per night. It often causes excessive daytime somnolence, and it may cause cardiovascular disease and brain damage. The syndrome is a common disorder, particularly in middle aged overweight males, although a person affected may have no awareness of the problem.

Other sleep related disorders include Cheyne-Stokes Respiration (CSR), Obesity Hyperventilation Syndrome (OHS) and Chronic Obstructive Pulmonary Disease (COPD). COPD encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors.

Continuous Positive Airway Pressure (CPAP) therapy has been used to treat Obstructive Sleep Apnea (OSA). Application of continuous positive airway pressure acts as a pneumatic splint and may prevent upper airway occlusion by pushing the soft palate and tongue forward and away from the posterior oropharyngeal wall.

Non-invasive ventilation (NIV) provides ventilatory support to a patient through the upper airways to assist the patient in taking a full breath and/or maintain adequate oxygen levels in the body by doing some or all of the work of breathing. The ventilatory support is provided via a user interface. NIV has been used to treat CSR, OHS, COPD, and Chest Wall disorders. In some forms, the comfort and effectiveness of these therapies may be improved. Invasive ventilation (IV) provides ventilatory support to patients that are no longer able to effectively breathe themselves and may be provided using a tracheostomy tube.

A treatment system (also identified herein as a respiratory therapy system) may comprise a Respiratory Pressure Therapy Device (RPT device), an air circuit, a humidifier, a user interface, and data management. A patient or user interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the user interface may form a seal, e.g., with a region of the patient's face, to facilitate the delivery of gas at a pressure at sufficient variance with ambient pressure to effect therapy, e.g., at a positive pressure of about 10 cm H2O relative to ambient pressure. For other forms of therapy, such as the delivery of oxygen, the user interface may not include a seal sufficient to facilitate delivery to the airways of a supply of gas at a positive pressure of about 10 cm H2O. Treatment of respiratory ailments by such therapy may be voluntary, and hence patients may elect not to comply with therapy if they find devices used to provide such therapy uncomfortable, difficult to use, expensive and/or aesthetically unappealing.

The design of a user interface presents a number of challenges. The face has a complex three-dimensional shape. The size and shape of noses varies considerably between individuals. Since the head includes bone, cartilage and soft tissue, different regions of the face respond differently to mechanical forces. The jaw or mandible may move relative to other bones of the skull. The whole head may move during the course of a period of respiratory therapy.

As a consequence of these challenges, some masks suffer from being one or more of obtrusive, aesthetically undesirable, costly, poorly fitting, difficult to use, and uncomfortable especially when worn for long periods of time or when a user is unfamiliar with a system. For example, masks designed solely for aviators, masks designed as part of personal protection equipment (e.g., filter masks), SCUBA masks, or for the administration of anesthetics may be tolerable for their original application, but nevertheless such masks may be undesirably uncomfortable to be worn for extended periods of time, e.g., several hours. This discomfort may lead to a reduction in user compliance with therapy. This is even more so if the mask is to be worn during sleep.

CPAP therapy is highly effective to treat certain respiratory disorders, provided users comply with therapy. Obtaining a user interface allows a user to engage in positive pressure therapy. Users seeking their first user interface or a new user interface to replace an older interface, typically consult a durable medical equipment provider to determine a recommended user interface size based on measurements of the user's facial anatomy, which are typically performed by the durable medical equipment provider. If a mask is uncomfortable, or difficult to use a user may not comply with therapy. Since it is often recommended that a user regularly wash their mask, if a mask is difficult to clean (e.g., difficult to assemble or disassemble), users may not clean their mask and this may impact on user compliance. In order for the air pressure therapy to effective, not only must comfort be provided to a user in wearing the mask, but a solid seal must be created between the face and the mask to minimize air leaks.

User interfaces, as described above, may be provided to a user in various forms, such as a nasal mask or full-face mask/oro-nasal mask (FFM) or nasal pillows mask, for example. Such user interfaces are manufactured with various dimensions to accommodate a specific user's anatomical features in order to facilitate a comfortable interface that is functional to provide, for example, positive pressure therapy. Such user interface dimensions may be customized to correspond with a particular user's specific facial anatomy or may be designed to accommodate a population of individuals that have an anatomy that falls within predefined spatial boundaries or ranges. However, in some cases masks may come in a variety of standard sizes from which a suitable one must be chosen.

In this regard, sizing a user interface for a user is typically performed by a trained individual, such as a Durable Medical Equipment (DME) provider or physician. Typically, a user needing a user interface to begin or continue positive pressure therapy would visit the trained individual at an accommodating facility where a series of measurements are made in an effort to determine an appropriate user interface size from standard sizes. An appropriate size is intended to mean a particular combination of dimensions of certain features, such as the seal forming structure, of a user interface, which provide adequate comfort and sealing to effectuate positive pressure therapy. Sizing in this way is not only labor intensive but also inconvenient. The inconvenience of taking time out of a busy schedule or, in some instances, having to travel great distances is a barrier to many users receiving a new or replacement user interface and ultimately a barrier to receiving treatment. This inconvenience prevents users from receiving a needed user interface and from engaging in positive pressure therapy. Nevertheless, selection of the most appropriate size is important for treatment quality and compliance.

There is a need for a system that allows for an accurate individualized fit for a user interface based on selected facial dimension data. There is a need for a system that incorporates data relating to other similar users using respiratory therapy devices to further select an interface that provides comfort in use for respiratory therapy. There is another need for the selection of an interface that minimizes leaks between the interface and the face of the user.

### SUMMARY

The disclosed system provides an adaptable system to size masks for use with a respiratory therapy device for better compliance with therapy for individual users. The system collects facial data from a primary user and also respiratory therapy device use and other data from a larger population of users to assist in the selection of an optimal mask for the primary user.

One disclosed example is a system to select an interface suited to a face of a user for respiratory therapy. The system includes a storage device storing a facial image of the user. A facial profile engine is operable to determine facial features based on the facial image. One or more databases store a plurality of facial features from a user population and a corresponding plurality of interfaces. One or more databases store operational data of respiratory therapy devices used by the user population with the plurality of corresponding interfaces. A selection engine is coupled to the databases. The selection engine is operative to select an interface for the user from the plurality of corresponding interfaces based on a desired outcome from the stored operational data and the determined facial features.

Another disclosed example is a method to select a suitable interface to the face of a user for respiratory therapy. A facial image of the user is stored in a storage device. Facial dimensions are determined based on the landmarks. A plurality of facial features is stored from a user population and a corresponding plurality of interfaces used by the patient population is stored in one or more databases. Operational data of respiratory therapy devices used by the user population with the plurality of corresponding interfaces are stored in the one or more databases. An interface for the user is selected from the plurality of corresponding interfaces based on a desired outcome from the stored operational data and the determined facial features.

According to some implementations, an example method includes receiving sensor data associated with a current fit of an interface on a face of a user. The interface can be couplable to a respiratory device. The sensor data is collected by one or more sensors of a mobile device that may be separate from the respiratory device. The method also includes generating a face mapping using the sensor data. The face mapping is indicative of one or more features of the face of the user. The method also includes identifying, using the sensor data and the face mapping, a characteristic associated with the current fit. The characteristic is indicative of a quality of the current fit. The characteristic is associated with a characteristic location on the face mapping. The method also includes generating output feedback based on the identified characteristic and the characteristic location. The output feedback can be generated to evaluate or improve the current fit.

According to some implementations, an example system includes an electronic interface, a memory, and a control system. The electronic interface is configured to receive data associated with a current fit of an interface. The memory stores machine-readable instructions. The control system includes one or more processors configured to execute the machine-readable instructions to generate a face mapping using the received data and identify a characteristic associated with the current fit based on the received data and the face mapping. The control system is further configured to generate output feedback based on the identified characteristic. The output feedback can be generated to evaluate or improve the current fit.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims.

### BRIEF DESCRIPTION OF THE DRAWINGS

The disclosure will be better understood from the following description of exemplary embodiments together with reference to the accompanying drawings, in which:

FIG. 1 shows a system including a user wearing a user interface in the form of a full-face mask to receive PAP therapy from an example respiratory pressure therapy device;

FIG. 2 shows a user interface in the form of a nasal mask with headgear in accordance with one form of the present technology;

FIG. 3A is a front view of a face with several features of surface anatomy;

FIG. 3B is a side view of a head with several features of surface anatomy identified;

FIG. 3C is a base view of a nose with several features identified;

FIG. 4A shows a respiratory pressure therapy device in accordance with one form of the present technology;

FIG. 4B is a schematic diagram of the pneumatic path of a respiratory pressure therapy device in accordance with one form of the present technology;

FIG. 4C is a schematic diagram of the electrical components of a respiratory pressure therapy device in accordance with one form of the present technology;

FIG. 4D is a schematic diagram of the main data processing components of a respiratory pressure therapy system in accordance with one form of the present technology;

FIG. 5 is a block diagram of the placement of an acoustic sensor in the hose that supplies air to the user interface in FIG. 2;

FIG. 6 is a diagram of the components of a computing device used to capture facial data;

FIG. 7 is a diagram of an example system for automatically selecting a patient interface, the system including a computing device;

FIG. 8A is an example facial scan that shows different landmark points to identify facial dimensions for mask sizing;

FIG. 8B is a view of the facial scan in FIG. 8A that shows different landmark points to identify a first facial measurement;

FIG. 8C is a view of the facial scan in FIG. 8A that shows different landmark points to identify a second facial measurement;

FIG. 8D is a view of the facial scan in FIG. 8A that shows different landmark points to identify a third facial measurement;

FIG. 9 is flow diagram of the process of mask selection for a user based on scanning and analysis of user input in view of big data collected from a user database;

FIG. 10 is a flow diagram of the process of follow up evaluation from an initial selection of a mask to adjust correlation parameters from data relating to the primary user;

FIG. 11 is a perspective view of a user controlling a user device to collect sensor data associated with a current fit of a user interface;

FIG. 12 is a user's view of a user device being used to identify a thermal characteristic associated with a current fit of a user interface;

FIG. 13 is a user's view of a user device being used to identify a contour-based characteristic associated with a current fit of a user interface;

FIG. 14 is a flowchart depicting a process for evaluating fit of a user interface across user interface transition events; and

FIG. 15 is a flowchart depicting a process for evaluating fit of a user interface.

The present disclosure is susceptible to various modifications and alternative forms. Some representative embodiments have been shown by way of example in the drawings and will be described in detail herein. It should be understood, however, that the invention is not intended to be limited to the particular forms disclosed. Rather, the disclosure is to cover all modifications, equivalents, and alternatives falling within the spirit and scope of the invention as defined by the appended claims.

### DETAILED DESCRIPTION OF THE ILLUSTRATED EMBODIMENTS

The present inventions can be embodied in many different forms. Representative embodiments are shown in the drawings, and will herein be described in detail. The present disclosure is an example or illustration of the principles of the present disclosure, and is not intended to limit the broad aspects of the disclosure to the embodiments illustrated. To that extent, elements and limitations that are disclosed, for example, in the Abstract, Summary, and Detailed Description sections, but not explicitly set forth in the claims, should not be incorporated into the claims, singly or collectively, by implication, inference, or otherwise. For purposes of the present detailed description, unless specifically disclaimed, the singular includes the plural and vice versa; and the word “including” means “including without limitation.” Moreover, words of approximation, such as “about,” “almost,” “substantially,” “approximately,” and the like, can be used herein to mean “at,” “near,” or “nearly at,” or “within 3-5% of,” or “within acceptable manufacturing tolerances,” or any logical combination thereof, for example.

The present disclosure relates to a system and method for selecting an optimal interface for a user using a respiratory therapy device. The system collects individual facial feature data from a user from a facial scanning application on a mobile device, and generates a 3D model of the face from the scanned image. The system pinpoints key landmarks on the scanned image for determining different facial dimensions. The system collects data from multiple users to learn the correlation between different masks and successful respiratory therapy based on user data and operational data from respiratory therapy devices using the different masks. The system uses the facial dimension data and the collected data from multiple users to select a suitable mask size and type for the user in comparison with past successful matching based on the data collected from multiple users using interfaces and respiratory therapy devices. This data may also be used for further learning of new insights that can be provided to users to increase compliance/adherence to therapy. Thus, the method combines suitable facial scanning methods with landmark identification, mask selection, therapy monitoring and feedback to provide effective user interface selection.

Certain aspects and features of the present disclosure relate to evaluating and improving the fit of a user interface (e.g., a filtering facemask or a user interface of a respiratory therapy device). Sensor data from one or more sensors of a user device (e.g., portable user device, such as a smart phone, also known as a mobile computing device or mobile device) can be leveraged to help a user ensure that a wearable user interface (e.g., user interface to be used with a respiratory device) is properly fit to the user's face. The sensor(s) can collect data about the user's face i) before donning of the user interface; ii) while the user interface is worn; and/or iii) after removal of the user interface. A face mapping can be generated, identifying one or more features of the user's face. The sensor data and face mapping can then be leveraged to identify characteristics associated with the fit of the user interface, which are usable to generate output feedback to evaluate and/or improve the fit.

In other implementations of the disclosed example system, the interface is a mask. In another implementation, the respiratory therapy device is configured to provide one or more of a Positive Airway Pressure (PAP) or a Non-invasive ventilation (NIV). In another implementation, at least one of the respiratory therapy devices includes an audio sensor to collect audio data during the operation of the at least one of the respiratory therapy devices. In another implementation, the selection engine is operable to analyze the audio data to determine a type of the corresponding interface of the at least one of the respiratory therapy devices based on matching the audio data to an acoustic signature of a known interface. In another implementation, the selection engine selects the interface based on demographic data of the user compared with the demographic data of a population of users stored in one or more databases. In another implementation, the operational data includes one of flow rate, motor speed, and therapy pressure. In another implementation, the facial image is determined from a scan from a mobile device including a camera. In another implementation, the mobile device includes a depth sensor. The camera is a 3D camera. The facial features are three-dimensional features derived from a meshed surface derived from the facial image. In another implementation, the facial image is a two-dimensional image including landmarks and the facial features are three-dimensional features derived from the landmarks. In another implementation, the facial image is one of a plurality of two-dimensional facial images. The facial features are three-dimensional features derived from a 3D morphable model adapted to match the facial images. In another implementation, the facial image includes landmarks relating at least one facial dimension. In another implementation, the facial dimensions outcome is a seal between the interface and a facial surface to prevent leaks. In another implementation, the desired outcome is user compliance with therapy. In another implementation, the system includes a mobile computing device operable to collect subjective data input from a user, and the selection of the interface is based at least partly on the subjective data. In another implementation, the system includes a machine learning module operable to determine types of operational data correlated with interfaces achieving the desired outcome. In another implementation, the selected interface includes one of a plurality of types of interfaces and one of a plurality of sizes of interfaces. In another implementation, the selection engine receives feedback from the user based on operating the selected interface and based on an undesirable result, selects another interface based on the desired outcome. In another implementation, the undesirable result is one of low user compliance with therapy, a high leak, or an unsatisfactory subjective result data.

In other implementations of the above described disclosed method to provide a suitable fitting interface, the interface is a mask. In another implementation, the respiratory therapy device is configured to provide one or more of a Positive Airway Pressure (PAP) or a Non-invasive ventilation (NIV). In another implementation, at least one of the respiratory therapy devices includes an audio sensor to collect audio data during the operation of the at least one of the respiratory therapy devices. In another implementation, the selection includes analyzing the audio data to determine a type of the corresponding interface of the at least one of the respiratory therapy devices based on matching the audio data to an acoustic signature of a known interface. In another implementation, the selection includes comparing demographic data of the user with the demographic data of a population of users stored in the one or more databases. In another implementation, the operational data includes one of flow rate, motor speed, and therapy pressure. In another implementation, the facial image is determined from a scan from a mobile device including a camera. In another implementation, the mobile device includes a depth sensor. The camera is a 3D camera, and the facial features are three-dimensional features derived from a meshed surface derived from the facial image. In another implementation, the facial image is a two-dimensional image including landmarks and the facial features are three-dimensional features derived from the landmarks. In another implementation, the facial image is one of a plurality of two-dimensional facial images. The facial features are three-dimensional features derived from a 3D morphable model adapted to match the facial images. In another implementation, the facial image includes landmarks relating to at least one facial dimension. In another implementation, the facial dimensions outcome is a seal between the interface and a facial surface to prevent leaks. In another implementation, the desired outcome is the user compliance with therapy. In another implementation, the method includes collecting subjective data input from a user by at least one mobile computing device, and the selection of the interface is based at least partly on the subjective data. In another implementation, the method includes determining types of operational data correlated with interfaces achieving the desired outcome via a machine learning module. In another implementation, the selected interface includes one of a plurality of types of interfaces and one of a plurality of sizes of interfaces. In another implementation, the method includes receiving feedback from the user based on operating the selected interface; and based on an undesirable result, selecting another interface based on the desired outcome. In another implementation, the undesirable result is one of low user compliance with therapy, a high leak, or an unsatisfactory subjective result data.

In other implementations of the above disclosed method to provide a custom fit on an interface to a user, the interface is fluidly couplable to a respiratory device. In another implementation, generating the output feedback includes determining a suggested action, which if implemented, would affect the characteristic to improve the current fit; and presenting the suggested action using an electronic interface of the mobile device. In another implementation, the sensor data includes infrared data from i) a passive thermal sensor; ii) an active thermal sensor; or iii) both i and ii. In another implementation, the method further includes receiving sensor data associated with a current fit of the selected interface on a face of a user. The sensor data is collected by one or more sensors of a mobile device. The method further includes generating a face mapping using the sensor data. The face mapping is indicative of one or more features of the face of the user. The method further includes identifying, using the sensor data and the face mapping, a characteristic associated with the current fit. The characteristic is indicative of a quality of the current fit, and the characteristic is associated with a characteristic location on the face mapping. The method further includes generating output feedback based on the identified characteristic and the characteristic location. In another implementation, the sensor data includes distance data that is indicative of one or more distances between the one or more sensors of the mobile device and the face of the user. In another implementation, the one or more sensors includes i) a proximity sensor; ii) an infrared-based dot matrix sensor; iii) a LiDAR sensor; iv) a MEMS micro-mirror projector-based sensor; or v) any combination of i to iv. In another implementation, the sensor data is collected i) prior to the user donning the interface; ii) when the user is wearing the interface with the current fit; iii) following the user removing the interface; or iii) any combination of i, ii and iii. In another implementation, the method further includes receiving initial sensor data. The initial sensor data is associated with the face of the user prior to donning the interface. The identifying the characteristic includes comparing the initial sensor data with the sensor data. In another implementation, the characteristic includes: i) a localized temperature on the face of the user; ii) a change in localized temperature on the face of the user; iii) a localized color on the face of the user; iv) a change in localized color on the face of the user; v) a localized contour on the face of the user; vi) a change in localized contour on the face of the user; vii) a localized contour on the interface; viii) a localized change on the interface; ix) a localized temperature on the interface; or x) any combination of i through ix. In another implementation, the characteristic includes i) a vertical position of the interface with respect to the one or more features of the face of the user; ii) a horizontal position of the interface with respect to the one or more features of the face of the user; iii) a rotational orientation of the interface with respect to the one or more features of the face of the user; iv) a distance between an identified feature of the interface with respect to the one or more features of the face of the user; or v) any combination of i through iv. In another implementation, the one or more sensors includes one or more directional sensors, and the sensor data includes directional sensor data from the one or more directional sensors. The receiving sensor data includes scanning the face of the user using mobile device as the mobile device is oriented such that the one or more directional sensors are directed towards the face of the user; and tracking a progress of the scanning of the face. A non-visible stimulus indicative of the progress of the scanning of the face is generated. In another implementation, the method further includes receiving motion data associated with movement of the mobile device; and applying the motion data to the sensor data to account for movement of the mobile device relative to the face of the user. In another implementation, the method further includes generating an interface mapping using the sensor data. The interface mapping is indicative of a relative position of one or more features of the interface with respect to the face mapping. Identifying the characteristic includes using the interface mapping. In another implementation, the one or more sensors includes a camera and the sensor data includes camera data. The method further includes receiving sensor calibration data collected by the camera as the camera is directed towards a calibration surface; and calibrating the camera data of the sensor data based on the sensor calibration data. In another implementation, the method further includes identifying, using the sensor data and the face mapping, an additional characteristic, the additional characteristic being associated with a possible future failure of the interface. The method further includes generating output feedback based on the identified additional characteristic. The output feedback being usable to reduce a likelihood that the possible future failure will occur or delay occurrence of the possible future failure. In another implementation, the method further includes accessing historical sensor data associated with one or more historical fits of the interface on the face of the user prior to receiving the sensor data. The identifying the characteristic further uses the historical sensor data. In another implementation, the method further includes generating a current fit score using the sensor data and the face mapping. The output feedback is generated to improve a subsequent fit score. In another implementation, receiving sensor data includes receiving audio data from one or more audio sensors. The identifying the characteristic includes identifying an unintentional leak using the audio data. In another implementation, the one or more sensors includes a camera, an audio sensor, and a thermal sensor. The sensor data includes camera data, audio data, and thermal data. The identifying the characteristic includes: identifying a potential characteristic using at least one of the camera data, the audio data, and the thermal data; and confirming the potential characteristic using at least one other of the camera data, the audio data, and the thermal data. In another implementation, the method further includes presenting a user instruction indicative of an action to be implemented by the user. The method further includes receiving a completion signal indicative that the user has implemented the action. A first portion of the sensor data is collected prior to receiving the completion signal and a second portion of the sensor data is collected after receiving the completion signal. The identifying the characteristic includes comparing the first portion of the sensor data with the second portion of the sensor data. In another implementation, generating the face mapping includes: identifying a first individual and a second individual using the received sensor data; identifying the first individual as being associated with the interface; and generating the face mapping for the first individual. In another implementation, receiving the sensor data includes determining adjustment data from the received sensor data. The adjustment data is associated with i) movements of the mobile device, ii) inherent noise of the mobile device, iii) breathing noise of the user, iv) speaking noise of the user, v) changes in ambient lighting, vi) detected transient shadows cast on the face of the user, vii) detected transient colored light cast on the face of the user, or viii) any combination of i to vii. The receiving the sensor data also includes applying adjustments to at least some of the received sensor data based on the adjustment data. In another implementation, receiving the sensor data includes: receiving image data associated with a camera of the one or more sensors, the camera operating in the visual light spectrum; receiving unstabilized data associated with an additional sensor of the one or more sensors, the additional sensor being an image sensor operating outside the visual light spectrum or a ranging sensor; determining image stabilization information associated with stabilization of the image data; and stabilizing the unstabilized data using the image stabilization information associated with stabilization of the image data. In another implementation, the output feedback is usable to improve the current fit. In another implementation, the method further includes generating an initial score based on the current fit using the sensor data. The method further includes receiving subsequent sensor data associated with a subsequent fit of the interface on the face of the user. The subsequent fit is based on the current fit after implementation of the output feedback. The method further includes generating a subsequent score based on the subsequent fit using the subsequent sensor data; and evaluating the subsequent score indicative of an improvement in quality over the initial score. In another implementation, identifying the characteristic associated with the current fit includes: determining a breathing pattern of the user based on the received sensor data; determining a thermal pattern associated with the face of the user based on the received sensor data and the face mapping; and determining a leak characteristic using the breathing pattern and the thermal pattern. The leak characteristic is indicative of a balance between intentional vent leak and unintentional seal leak. In another implementation, the one or more sensors include at least two sensors selected from the group consisting of: i) a passive thermal sensor; ii) an active thermal sensor; iii) a camera; iv) an accelerometer; v) a gyroscope; vi) an electronic compass; vii) a magnetometer; viii) a pressure sensor; ix) a microphone; x) a temperature sensor; xi) a proximity sensor; xii) an infrared-based dot matrix sensor; xiii) a LiDAR sensor; xiv) a MEMS micro-mirror projector-based sensor; xv) a radio frequency-based ranging sensor; and xvi) a wireless network interface. In another implementation, the method includes receiving additional sensor data from one or more additional sensors of the respiratory device. The identifying the characteristic includes using the additional sensor data. In another implementation, the method further includes transmitting a control signal which, when received by the respiratory device, causes the respiratory device to operate using a set of defined parameters. A first portion of the sensor data is collected while the respiratory device is operating using the set of defined parameters and a second portion of the sensor data is collected while the respiratory device is not operating using the set of defined parameters. Identifying the characteristic includes comparing the first portion of the sensor data with the second portion of the sensor data.

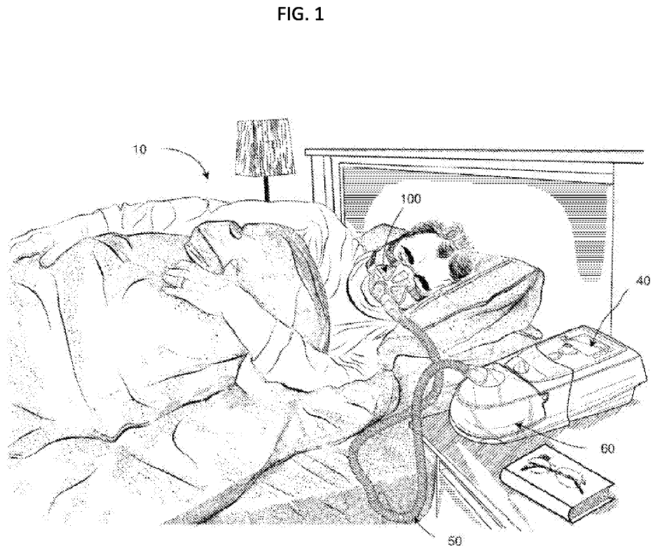

FIG. 1 shows a system including a user 10 wearing a user interface 100, in the form of a full-face mask (FFM), receives a supply of air at positive pressure from a respiratory therapy device such as a positive airway pressure (PAP) device and specifically a respiratory pressure therapy (RPT) device 40. Air from the RPT device 40 is humidified in a humidifier 60, and passes along an air circuit 50 to the user 10.

In this example, the respiratory therapy devices described herein may include any respiratory therapy device is configured to provide one or more of a Positive Airway Pressure (PAP), Non-invasive ventilation (NIV), or invasive ventilation. In this example, the PAP device may be a continuous positive airway pressure (CPAP) device, an automatic positive airway pressure device (APAP), a bi-level or variable positive airway pressure device (BPAP or VPAP), or any combination thereof. The CPAP device delivers a predetermined air pressure (e.g., determined by a sleep physician) to the user. The APAP device automatically varies the air pressure delivered to the user based on, for example, respiration data associated with the user. The BPAP or VPAP device is configured to deliver a first predetermined pressure (e.g., an inspiratory positive airway pressure or IPAP) and a second predetermined pressure (e.g., an expiratory positive airway pressure or EPAP) that is lower than the first predetermined pressure.

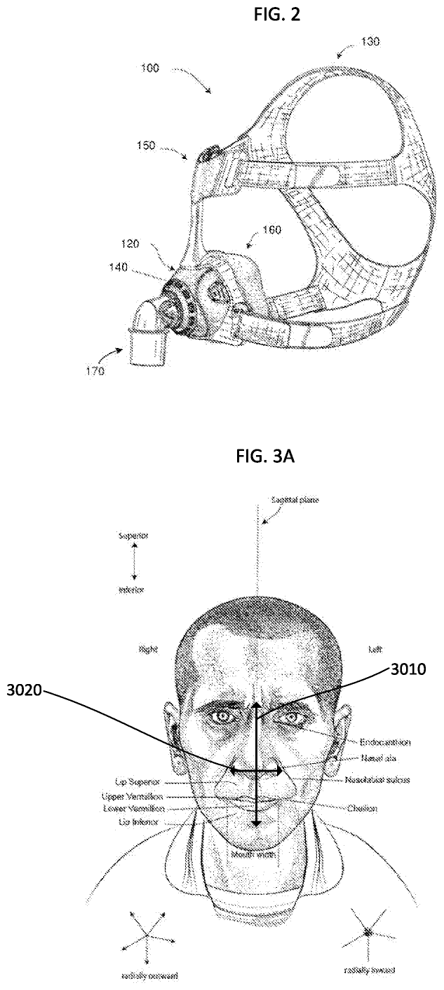

FIG. 2 depicts the user interface 100 in accordance with one aspect of the present technology comprises the following functional aspects: a seal-forming structure 160, a plenum chamber 120, a positioning and stabilising structure 130, a vent 140, a forehead support 150, one form of connection port 170 for connection to the air circuit 50 in FIG. 1. In some forms a functional aspect may be provided by one or more physical components. In some forms, one physical component may provide one or more functional aspects. In use the seal-forming structure 160 is arranged to surround an entrance to the airways of the user so as to facilitate the supply of air at positive pressure to the airways.

In one form of the present technology, a seal-forming structure 160 provides a seal-forming surface, and may additionally provide a cushioning function. The seal-forming structure 160 in accordance with the present technology may be constructed from a soft, flexible, resilient material such as silicone. In one form the seal-forming portion of the non-invasive user interface 100 comprises a pair of nasal puffs, or nasal pillows, each nasal puff or nasal pillow being constructed and arranged to form a seal with a respective naris of the nose of a user.

Nasal pillows in accordance the present technology include: a frusto-cone, at least a portion of which forms a seal on an underside of the user's nose, a stalk, a flexible region on the underside of the frusto-cone and connecting the frusto-cone to the stalk. In addition, the structure to which the nasal pillow of the present technology is connected includes a flexible region adjacent the base of the stalk. The flexible regions can act in concert to facilitate a universal joint structure that is accommodating of relative movement both displacement and angular of the frusto-cone and the structure to which the nasal pillow is connected. For example, the frusto-cone may be axially displaced towards the structure to which the stalk is connected.

In one form, the non-invasive user interface 100 comprises a seal-forming portion that forms a seal in use on an upper lip region (that is, the lip superior) of the user's face. In one form the non-invasive user interface 100 comprises a seal-forming portion that forms a seal in use on a chin-region of the user's face.

Preferably the plenum chamber 120 has a perimeter that is shaped to be complementary to the surface contour of the face of an average person in the region where a seal will form in use. In use, a marginal edge of the plenum chamber 120 is positioned in close proximity to an adjacent surface of the face. Actual contact with the face is provided by the seal-forming structure 160. The seal-forming structure 160 may extend in use about the entire perimeter of the plenum chamber 120.

Preferably the seal-forming structure 160 of the user interface 100 of the present technology may be held in sealing position in use by the positioning and stabilising structure 130.

In one form, the user interface 100 includes a vent 140 constructed and arranged to allow for the washout of exhaled carbon dioxide. One form of vent 140 in accordance with the present technology comprises a plurality of holes, for example, about 20 to about 80 holes, or about 40 to about 60 holes, or about 45 to about 55 holes.

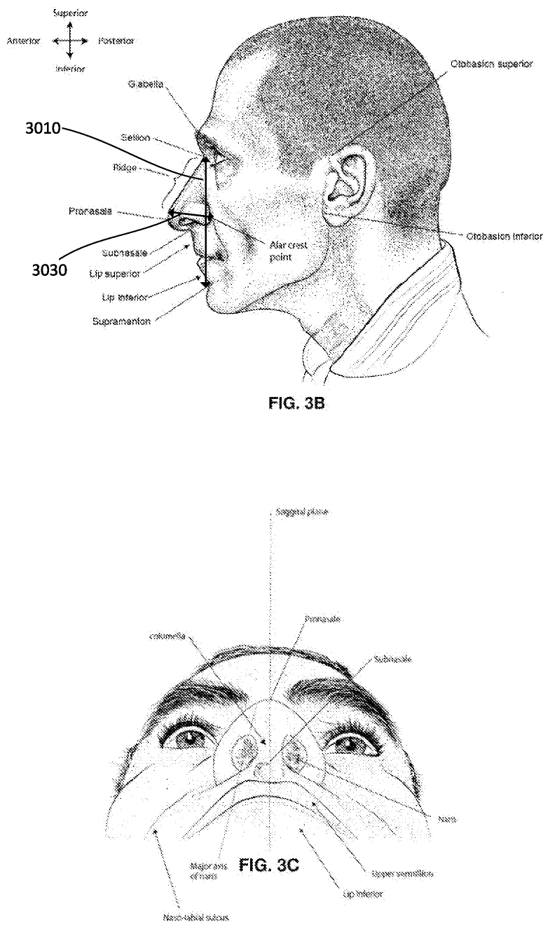

FIG. 3A shows an anterior view of a human face including the endocanthion, nasal ala, nasolabial sulcus, lip superior and inferior, upper and lower vermillion, and chelion. Also shown are the mouth width, the sagittal plane dividing the head into left and right portions, and directional indicators. The directional indicators indicate radial inward/outward and superior/inferior directions. FIG. 3B shows a lateral view of a human face including the glabaella, sellion, nasal ridge, pronasale, subnasale, superior and inferior lip, supramenton, alar crest point, and otobasion superior and inferior. Also shown are directional indictors indicating superior/inferior and anterior/posterior directions. FIG. 3C shows a base view of a nose with several features identified including naso-labial sulcus, lip inferior, upper Vermilion, naris, subnasale, columella, pronasale, the major axis of a naris and the sagittal plane.

The following are more detailed explanations of the features of the human face shown in FIGS. 3A-3C.

Ala: The external outer wall or “wing” of each nostril (plural: alar)

Alare: The most lateral point on the nasal ala.

Alar curvature (or alar crest) point: The most posterior point in the curved base line of each ala, found in the crease formed by the union of the ala with the cheek.

Auricle: The whole external visible part of the ear.

Columella: The strip of skin that separates the nares and which runs from the pronasale to the upper lip.

Columella angle: The angle between the line drawn through the midpoint of the nostril aperture and a line drawn perpendicular to the Frankfurt horizontal while intersecting subnasale.

Glabella: Located on the soft tissue, the most prominent point in the midsagittal plane of the forehead.

Nares (Nostrils): Approximately ellipsoidal apertures forming the entrance to the nasal cavity. The singular form of nares is naris (nostril). The nares are separated by the nasal septum.

Naso-labial sulcus or Naso-labial fold: The skin fold or groove that runs from each side of the nose to the corners of the mouth, separating the cheeks from the upper lip.

Naso-labial angle: The angle between the columella and the upper lip, while intersecting subnasale.

Otobasion inferior: The lowest point of attachment of the auricle to the skin of the face.

Otobasion superior: The highest point of attachment of the auricle to the skin of the face.

Pronasale: The most protruded point or tip of the nose, which can be identified in lateral view of the rest of the portion of the head.

Philtrum: The midline groove that runs from lower border of the nasal septum to the top of the lip in the upper lip region.

Pogonion: Located on the soft tissue, the most anterior midpoint of the chin.

Ridge (nasal): The nasal ridge is the midline prominence of the nose, extending from the Sellion to the Pronasale.

Sagittal plane: A vertical plane that passes from anterior (front) to posterior (rear) dividing the body into right and left halves.

Sellion: Located on the soft tissue, the most concave point overlying the area of the frontonasal suture.

Septal cartilage (nasal): The nasal septal cartilage forms part of the septum and divides the front part of the nasal cavity.

Subalare: The point at the lower margin of the alar base, where the alar base joins with the skin of the superior (upper) lip.

Subnasal point: Located on the soft tissue, the point at which the columella merges with the upper lip in the midsagittal plane.

Supramenton: The point of greatest concavity in the midline of the lower lip between labrale inferius and soft tissue pogonion.

As will be explained below, there are several critical dimensions from a face that may be used to select the sizing for a user interface such as the mask 100 in FIG. 1. In this example there are three dimensions including the face height, the nose width, and the nose depth. FIGS. 3A-3B show a line 3010 that represents the face height. As may be seen in FIG. 3A, the face height is the distance between the sellion to the supramenton. A line 3020 in FIG. 3A represents the nose width, which may be the distance between the alare (e.g. the left and right lateral-most points on the nasal alar) of the nose. A line 3030 in FIG. 3B represents the nose depth, which may be the distance between pronasale and alar crest points in a direction parallel to the sagittal plane.

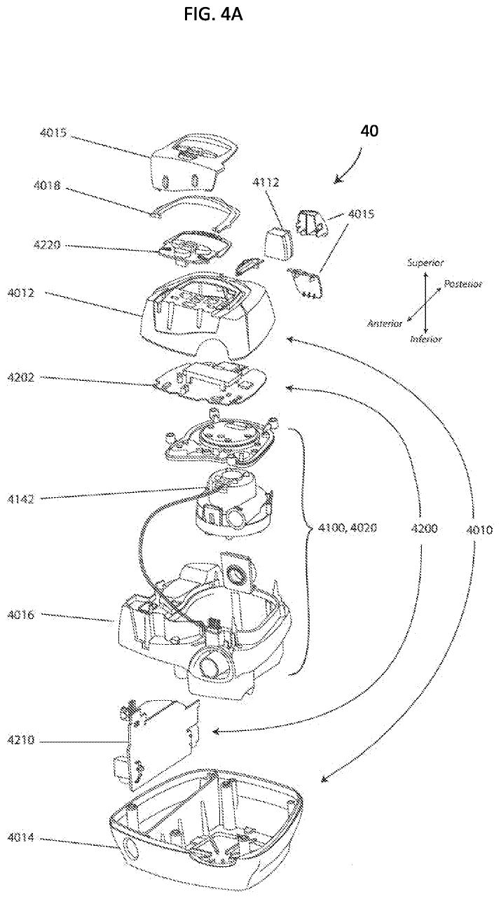

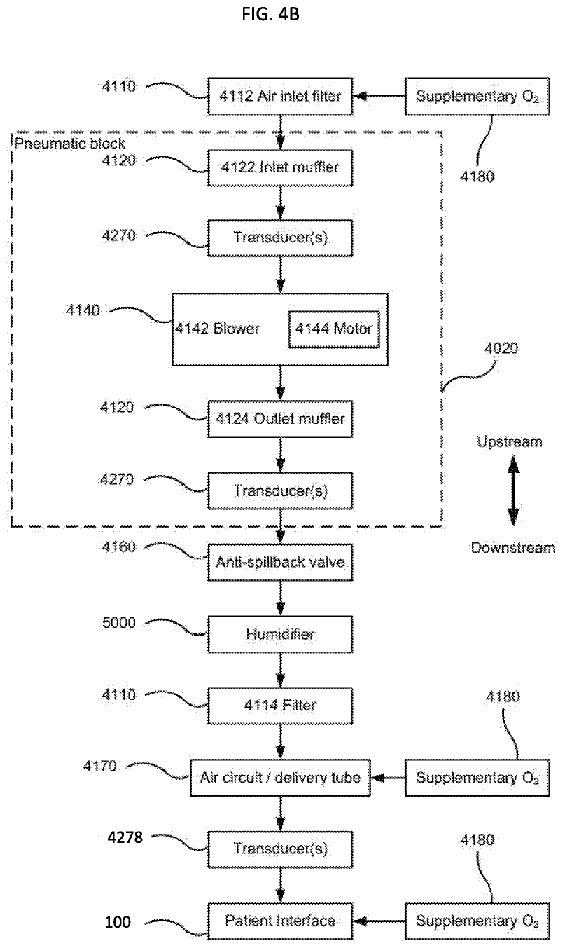

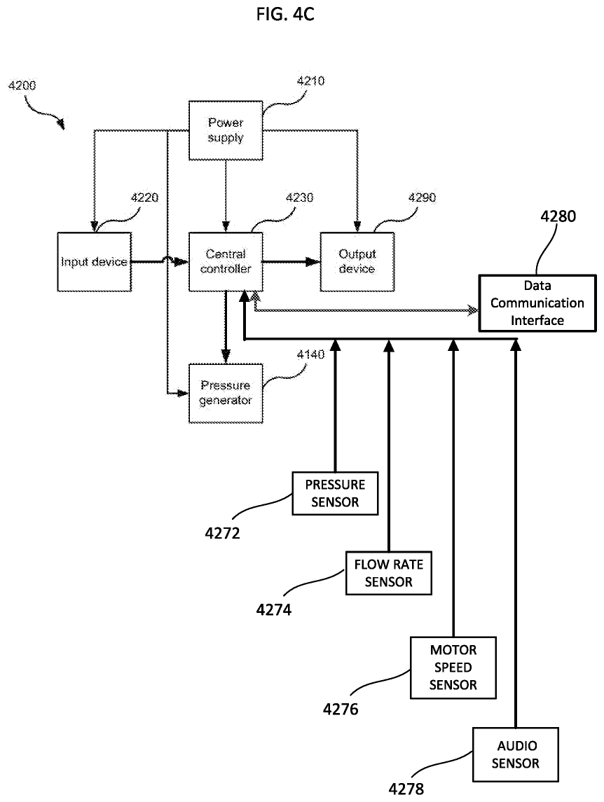

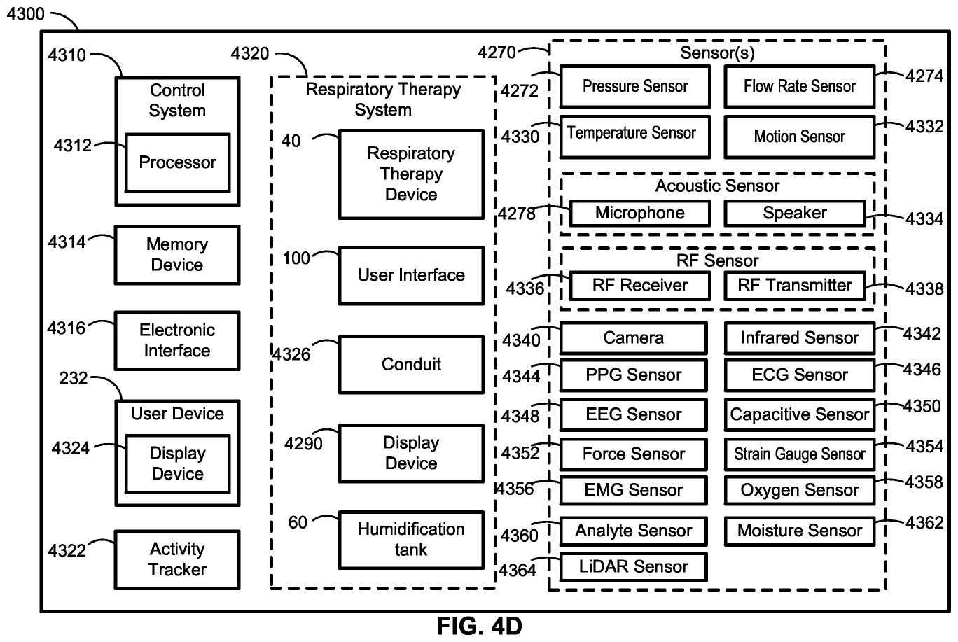

FIG. 4A shows an exploded view of the components of an example RPT device 40 in accordance with one aspect of the present technology comprises mechanical, pneumatic, and/or electrical components and is configured to execute one or more algorithms, such as any of the methods, in whole or in part, described herein. FIG. 4B shows a block diagram of the example RPT device 40. FIG. 4C shows a block diagram of the electrical control components of the example RPT device 40. The directions of upstream and downstream are indicated with reference to the blower and the user interface. The blower is defined to be upstream of the user interface and the user interface is defined to be downstream of the blower, regardless of the actual flow direction at any particular moment. Items which are located within the pneumatic path between the blower and the user interface are downstream of the blower and upstream of the user interface. The RPT device 40 may be configured to generate a flow of air for delivery to a user's airways, such as to treat one or more of the respiratory conditions.

The RPT device 40 may have an external housing 4010, formed in two parts, an upper portion 4012 and a lower portion 4014. Furthermore, the external housing 4010 may include one or more panel(s) 4015. The RPT device 40 comprises a chassis 4016 that supports one or more internal components of the RPT device 40. The RPT device 40 may include a handle 4018.

The pneumatic path of the RPT device 40 may comprise one or more air path items, e.g., an inlet air filter 4112, an inlet muffler 4122, a pressure generator 4140 capable of supplying air at positive pressure (e.g., a blower 4142), an outlet muffler 4124 and one or more transducers 4270, such as a pressure sensor 4272, a flow rate sensor 4274, and a motor speed sensor 4276.

One or more of the air path items may be located within a removable unitary structure which will be referred to as a pneumatic block 4020. The pneumatic block 4020 may be located within the external housing 4010. In one form a pneumatic block 4020 is supported by, or formed as part of the chassis 4016.

The RPT device 40 may have an electrical power supply 4210, one or more input devices 4220, a central controller 4230, a pressure generator 4140, a data communication interface 4280, and one or more output devices 4290. A separate controller may be provided for the therapy device. Electrical components 4200 may be mounted on a single Printed Circuit Board Assembly (PCBA) 4202. In an alternative form, the RPT device 40 may include more than one PCBA 4202. Other components such as the one or more protection circuits 4250, transducers 4270, the data communication interface 4280, and storage devices may also be mounted on the PCBA 4202.

An RPT device may comprise one or more of the following components in an integral unit. In an alternative form, one or more of the following components may be located as respective separate units.

An RPT device in accordance with one form of the present technology may include an air filter 4110, or a plurality of air filters 4110. In one form, an inlet air filter 4112 is located at the beginning of the pneumatic path upstream of a pressure generator 4140. In one form, an outlet air filter 4114, for example an antibacterial filter, is located between an outlet of the pneumatic block 4020 and a user interface 100.

An RPT device in accordance with one form of the present technology may include a muffler 4120, or a plurality of mufflers 4120. In one form of the present technology, an inlet muffler 4122 is located in the pneumatic path upstream of a pressure generator 4140. In one form of the present technology, an outlet muffler 4124 is located in the pneumatic path between the pressure generator 4140 and a user interface 100 in FIG. 1.

In one form of the present technology, a pressure generator 4140 for producing a flow, or a supply, of air at positive pressure is a controllable blower 4142. For example, the blower 4142 may include a brushless DC motor 4144 with one or more impellers. The impellers may be located in a volute. The blower may be capable of delivering a supply of air, for example at a rate of up to about 120 litres/minute, at a positive pressure in a range from about 4 cm H2O to about 20 cm H2O, or in other forms up to about 30 cm H2O. The blower may be as described in any one of the following patents or patent applications the contents of which are incorporated herein by reference in their entirety: U.S. Pat. Nos. 7,866,944; 8,638,014; 8,636,479; and PCT Patent Application Publication No. WO 2013/020167.

The pressure generator 4140 is under the control of the therapy device controller 4240. In other forms, a pressure generator 4140 may be a piston-driven pump, a pressure regulator connected to a high pressure source (e.g. compressed air reservoir), or a bellows.

An air circuit 4170 in accordance with an aspect of the present technology is a conduit or a tube constructed and arranged to allow, in use, a pressurized flow of air to travel between two components such as the humidifier 60 and the user interface 100. In particular, the air circuit 4170 may be in fluid communication with the outlet of the humidifier 60 and the plenum chamber 120 of the user interface 100.

In one form of the present technology, an anti-spill back valve 4160 is located between the humidifier 60 and the pneumatic block 4020. The anti-spill back valve is constructed and arranged to reduce the risk that water will flow upstream from the humidifier 60, for example to the motor 4144.

A power supply 4210 may be located internal or external of the external housing 4010 of the RPT device 40. In one form of the present technology, power supply 4210 provides electrical power to the RPT device 40 only. In another form of the present technology, power supply 4210 provides electrical power to both RPT device 40 and humidifier 60.

An RT system may comprise one or more transducers (sensors) 4270 configured to measure one or more of any number of parameters in relation to an RT system, its user, and/or its environment. A transducer may be configured to produce an output signal representative of the one or more parameters that the transducer is configured to measure.

The output signal may be one or more of an electrical signal, a magnetic signal, a mechanical signal, a visual signal, an optical signal, a sound signal, or any number of others which are known in the art.

A transducer may be integrated with another component of an RT system, where one exemplary arrangement would be the transducer being internal of an RPT device. A transducer may be substantially a ‘standalone’ component of an RT system, an exemplary arrangement of which would be the transducer being external to the RPT device.

A transducer may be configured to communicate its output signal to one or more components of an RT system, such as an RPT device, a local external device, or a remote external device. External transducers may be for example located on a user interface, or in an external computing device, such as a smart phone. External transducers may be located for example on or form part of the air circuit, e.g., the user interface.

FIG. 4D shows a system 4300, according to some implementations of the present disclosure, is illustrated. The system 4300 includes a control system 4310 that includes a processor 4312, a memory device 4314, an electronic interface 4316, and one or more sensors 4270. In some implementations, the system 4300 further optionally includes a respiratory therapy system 4320 that may be the system in FIG. 2 that includes the RPT device 40, a user device such as a mobile device 234, and an activity tracker 4322. The device 234 may include a display 4344. In some cases, some or most of system 4300 can be implemented as the mobile device 232, the RPT device 40, or in other external devices such as external computing devices. As explained above, the respiratory therapy system 4320 includes the RPT device 40, the user or user interface 100, a conduit 4326, a display such as the output 4290, and the humidifier 60.

The one or more sensors or transducers 4270 may be constructed and arranged to generate signals representing properties of air such as a flow rate, a pressure or a temperature. The air may be a flow of air from the RPT device to a user, a flow of air from the user to the atmosphere, ambient air or any others. The signals may be representative of properties of the flow of air at a particular point, such as the flow of air in the pneumatic path between the RPT device and the user. In one form of the present technology, one or more transducers 4270 are located in a pneumatic path of the RPT device, such as downstream of the humidifier 60.

In accordance with one aspect of the present technology, the one or more transducers 4270 comprises a pressure sensor located in fluid communication with the pneumatic path. An example of a suitable pressure sensor is a transducer from the HONEYWELL ASDX series. An alternative suitable pressure sensor is a transducer from the NPA Series from GENERAL ELECTRIC. In one implementation, the pressure sensor is located in the air circuit 4170 adjacent the outlet of the humidifier 60.

An acoustic/audio sensor 4278 (such as a microphone pressure sensor 4278) is configured to generate a sound signal representing the variation of pressure within the air circuit 4170. The “acoustic sensor” or “audio sensor” are interchangeable terms and relate to a sensor that can detect sounds that are audible and/or inaudible to a human. The sound signal from the audio sensor 4278 may be received by the central controller 4230 for acoustic processing and analysis as configured by one or more of the algorithms described below. The audio sensor 4278 may be directly exposed to the airpath for greater sensitivity to sound, or may be encapsulated behind a thin layer of flexible membrane material. This membrane may function to protect the audio sensor 4278 from heat and/or humidity. Alternatively, the audio sensor 4278 can be coupled to or integrated in the RPT device 40, the user interface 100, or the conduit or an external user device.

The audio data generated by the audio sensor 4278 is reproducible as one or more sound(s) (e.g., sounds from the user 10). The audio data form the audio sensor 4278 can also be used to identify (e.g., using the central controller 4230) or confirm characteristics associated with a user interface, such as the sound of air escaping a valve.

A speaker may output sound waves that are audible to a user such as the user 10. The speaker can be used, for example, to provide audio feedback, such as to indicate how to manipulate the RPT device 40 or another device to achieve desirable sensor data, or to indicate when the collection of sensor data is sufficiently complete. In some implementations, the speaker can be used to communicate the audio data generated by the audio sensor 4278 to the user. The speaker can be coupled to or integrated in the RPT device 40, the user interface 100, the conduit, or an external device.

The audio sensor 4278 and the speaker can be used as separate devices. In some implementations, the audio sensor 4278 and the speaker can be combined into an acoustic sensor (e.g., a SONAR sensor), as described in, for example, WO 2018/050913 and WO 2020/104465, each of which is hereby incorporated by reference herein in its entirety. In such implementations, the speaker generates or emits sound waves at a predetermined interval and the audio sensor 4278 detects the reflections of the emitted sound waves from the speaker. The sound waves generated or emitted by the speaker have a frequency that is not audible to the human ear (e.g., below 20 Hz or above around 18 kHz) so as not to disturb the user 10. Based at least in part on the data from the audio sensor 4278 and/or the speaker, a control system can determine location information pertaining to the user and/or the user interface 100 (e.g., a location of the user's face, a location of features on the user's face, a location of the user interface 100, a location of features on the user interface 100), physiological parameters (e.g., respiration rate), and the like. In such a context, a sonar sensor may be understood to concern an active acoustic sensing, such as by generating and/or transmitting ultrasound and/or low frequency ultrasound sensing signals (e.g., in a frequency range of about 17-23 kHz, 18-22 kHz, or 17-18 kHz, for example), through the air. Such a system may be considered in relation to WO 2018/050913 and WO 2020/104465 mentioned above, each of which is hereby incorporated by reference herein in its entirety. In some implementations, additional microphone pressure sensors may be used.

Data from the transducers 4270 such as the pressure sensor 4272, flow rate sensor 4274, motor speed sensor 4276, and audio sensor 4278 may be collected by central controller 4230 on a periodic basis. Such data generally relates to the operational state of the RPT device 40. In this example, the central controller 4230 encodes such data from the sensors in a proprietary data format. The data may also be coded in a standardized data format.

The one or more sensors or transducers 4270 may also include a temperature sensor 4330, a motion sensor 4332, a speaker 4334, a radio-frequency (RF) receiver 4336, a RF transmitter 4338, a camera 4340, an infrared sensor 4342 (e.g., a passive infrared sensor or an active infrared sensor), a photoplethysmogram (PPG) sensor 4344, an electrocardiogram (ECG) sensor 4346, an electroencephalography (EEG) sensor 4348, a capacitive sensor 4350, a force sensor 4352, a strain gauge sensor 4354, an electromyography (EMG) sensor 4356, an oxygen sensor 4358, an analyte sensor 4360, a moisture sensor 4362, a LiDAR sensor 4364, or any combination thereof. Generally, each of the one or more sensors or transducers 4270 are configured to output sensor data that is received and stored in a memory device.

While the one or more sensors 4270 are shown and described as including each of the pressure sensor 4272, the flow rate sensor 4274, motor speed sensor 4276, and audio sensor 4278, other sensors as noted above can include any combination and any number of each of the sensors described and/or shown herein.

The one or more sensors 4270 can be used to generate, sensor data, such as image data, audio data, rangefinding data, contour mapping data, thermal data, physiological data, ambient data, and the like. The sensor data can be used by a control system to determine a user interface and identify characteristics associated with a current fit of the user interface 100.

The example pressure sensor 4272 is an air pressure sensor (e.g., barometric pressure sensor) that generates sensor data indicative of the respiration (e.g., inhaling and/or exhaling) of the user of the respiratory therapy system in FIG. 1 and/or ambient pressure. In such implementations, the pressure sensor 4272 can be coupled to or integrated in the RPT device 40. The pressure sensor 4272 can be, for example, a capacitive sensor, an electromagnetic sensor, a piezoelectric sensor, a strain-gauge sensor, an optical sensor, a potentiometric sensor, or any combination thereof.

Examples of flow rate sensors (such as, for example, the flow rate sensor 4274) are described in WO 2012/012835, which is hereby incorporated by reference herein in its entirety. In some implementations, the flow rate sensor 4274 is used to determine an air flow rate from the RPT device 40, an air flow rate through the conduit of the air circuit 4170, an air flow rate through the user interface 100, or any combination thereof. In such implementations, the flow rate sensor 4274 can be coupled to or integrated in the RPT device 40, the user interface 100, or the conduit attaching the user interface 100 to the RPT device 40. The flow rate sensor 4274 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof. In some implementations, the flow rate sensor 4274 is configured to measure a vent flow (e.g., intentional “leak”), an unintentional leak (e.g., mouth leak and/or mask leak), a user flow (e.g., air into and/or out of lungs), or any combination thereof. In some implementations, the flow rate data can be analyzed to determine cardiogenic oscillations of the user.

In some implementations, the temperature sensor 4330 generates temperatures data indicative of a core body temperature of the user 10, a localized or average skin temperature of the user, a localized or average temperature of the air flowing from the RPT device 40 and/or through the conduit, a localized or average temperature in the user interface 100, an ambient temperature, or any combination thereof. The temperature sensor 4330 can be, for example, a thermocouple sensor, a thermistor sensor, a silicon band gap temperature sensor or semiconductor-based sensor, a resistance temperature detector, or any combination thereof. In some cases, the temperature sensor is a non-contact temperature sensor, such as an infrared pyrometer.

The RF transmitter 4338 generates and/or emits radio waves having a predetermined frequency and/or a predetermined amplitude (e.g., within a high frequency band, within a low frequency band, long wave signals, short wave signals, etc.). The RF receiver 4336 detects the reflections of the radio waves emitted from the RF transmitter 4338, and this data can be analyzed by a control system to determine location information pertaining to the user 10 and/or user interface 100, and/or one or more of the physiological parameters described herein. An RF receiver (either the RF receiver and the RF transmitter or another RF pair) can also be used for wireless communication between external components and the RPT device 40, or any combination thereof. The RF receiver 4336 and RF transmitter 4338 may be combined as a part of an RF sensor (e.g., a RADAR sensor). In some such implementations, the RF sensor includes a control circuit. The specific format of the RF communication can be WiFi, Bluetooth, or the like.

In some implementations, the RF sensor is a part of a mesh system. One example of a mesh system is a WiFi mesh system, which can include mesh nodes, mesh router(s), and mesh gateway(s), each of which can be mobile/movable or fixed. In such implementations, the WiFi mesh system includes a WiFi router and/or a WiFi controller and one or more satellites (e.g., access points), each of which include an RF sensor that the is the same as, or similar to, the RF sensor. The WiFi router and satellites continuously communicate with one another using WiFi signals. The WiFi mesh system can be used to generate motion data based on changes in the WiFi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The image data from the camera 4340 can be used by a control system to determine information associated with the face of the user, the user interface 100, and/or one or more of the physiological parameters described herein. For example, the image data from the camera 4340 can be used to identify a location of the user, a localized color of a portion of the user's face, a relative location of a feature on the user interface with respect to a feature on the user's face, or the like. In some implementations, the camera includes a wide angle lens or a fish eye lens. The camera can be a camera that operates in the visual spectrum, such as at wavelengths between at or approximately 380 nm and at or approximately 740 nm.

The IR sensor 4342 can be a passive sensor or an active sensor. A passive IR sensor can measure natural infrared emissions or reflections from distant surfaces, such as measuring IR energy radiating from a surface to determine the surface's temperature. An active IR sensor can include an IR emitter that generates an IR signal, which is then received by an IR receiver. Such an active IR sensor can be used to measure IR reflection off and/or transmission through an object. For example, an IR emitter that is a dot projector can project a recognizable array of dots onto a user's face using IR light, the reflections of which can then be detected by an IR receiver to determine ranging data (e.g., data associated with a distance between the IR sensor 4342 and a distant surface, such as portion of the user's face) or contour data (e.g., data associated with relative heights features of a surface with respect to a nominal height of the surface)) associated with the user's face.

Generally, the infrared data from the IR sensor 4342 can be used to determine information pertaining to the user 10 and/or interface 100, and/or one or more of the physiological parameters described herein. In an example, the infrared data form the IR sensor can be used to detect localized temperatures on a portion of the user's face or a portion of the interface 100. The IR sensor 4342 can also be used in conjunction with the camera, such as to correlate IR data (e.g., temperature data or ranging data) with camera data (e.g., localized colors). The IR sensor 4342 can detect infrared light having a wavelength between at or approximately 700 nm and at or approximately 1 mm.

The PPG sensor 4344 outputs physiological data associated with the user 10 that can be used to determine one or more sleep-related parameters, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 4344 can be worn by the user, embedded in clothing and/or fabric that is worn by the user, embedded in and/or coupled to the interface 100 and/or its associated headgear (e.g., straps, etc.), etc.

The ECG sensor 4346 outputs physiological data associated with electrical activity of the heart of the user 10. In some implementations, the ECG sensor 4346 includes one or more electrodes that are positioned on or around a portion of the user 10 during the sleep session. The physiological data from the ECG sensor 4346 can be used, for example, to determine one or more of the sleep-related parameters described herein.

The EEG sensor 4348 outputs physiological data associated with electrical activity of the brain of the user 10. In some implementations, the EEG sensor 4348 includes one or more electrodes that are positioned on or around the scalp of the user 10 during the sleep session. The physiological data from the EEG sensor 4348 can be used, for example, to determine a sleep state and/or a sleep stage of the user 10 at any given time during the sleep session. In some implementations, the EEG sensor 4348 can be integrated in the interface 100 and/or the associated headgear (e.g., straps, etc.).

The EMG sensor 4356 outputs physiological data associated with electrical activity produced by one or more muscles. The oxygen sensor 4358 outputs oxygen data indicative of an oxygen concentration of gas (e.g., in the conduit or at the interface 100). The oxygen sensor 4358 can be, for example, an ultrasonic oxygen sensor, an electrical oxygen sensor, a chemical oxygen sensor, an optical oxygen sensor, a pulse oximeter (e.g., SpO2 sensor), or any combination thereof. In some implementations, the one or more sensors 4270 also include a galvanic skin response (GSR) sensor, a blood flow sensor, a respiration sensor, a pulse sensor, a sphygmomanometer sensor, an oximetry sensor, or any combination thereof.

The analyte sensor 4360 can be used to detect the presence of an analyte, such as in the exhaled breath of the user 10. The data output by the analyte sensor 4360 may be used by a control system to determine the identity and concentration of any analytes, such as in the breath of the user 10. In some implementations, the analyte sensor is positioned near a mouth of the user 10 to detect analytes in breath exhaled from the mouth of the user 10. For example, when the interface 100 is a facial mask that covers the nose and mouth of the user 10, the analyte sensor 4360 can be positioned within the facial mask to monitor mouth breathing of the user 10. In other implementations, such as when the interface 100 is a nasal mask or a nasal pillow mask, the analyte sensor can be positioned near the nose of the user 10 to detect analytes in breath exhaled through the nose. In still other implementations, the analyte sensor can be positioned near the mouth when the interface 100 is a nasal mask or a nasal pillow mask. In this implementation, the analyte sensor 4360 can be used to detect whether any air is inadvertently leaking from the mouth of the user 10. In some implementations, the analyte sensor 4360 is a volatile organic compound (VOC) sensor that can be used to detect carbon-based chemicals or compounds. In some implementations, the analyte sensor 4360 can also be used to detect whether the user 10 is breathing through their nose or mouth. For example, if the data output by an analyte sensor positioned near the mouth or within the facial mask (in implementations where the interface e100 is a facial mask) detects the presence of an analyte, a control system can use this data as an indication that the user 10 is breathing through their mouth.

The moisture sensor 4362 can be used to detect moisture in various areas surrounding the user 10 (e.g., inside the conduit or the interface 100, near the face of the user 10, near the connection between the conduit and the interface 100, near the connection between the conduit and the RPT device 40, etc.). Thus, in some implementations, the moisture sensor 4362 can be coupled to or integrated in the interface 100 or in the conduit to monitor the humidity of the pressurized air from the RPT device 40. In other implementations, the moisture sensor 4362 is placed near any area where moisture levels need to be monitored. The moisture sensor 4362 can also be used to monitor the humidity of the ambient environment surrounding the user 10, for example, the air inside the bedroom.

The Light Detection and Ranging (LiDAR) sensor 4364 can be used for depth sensing. This type of optical sensor (e.g., laser sensor) can be used to detect objects and build three dimensional (3D) maps (e.g., contour maps) of objects such as the user's face, the interface 100, or the surroundings (e.g., a living space). LiDAR can generally utilize a pulsed laser to make time of flight measurements. LiDAR is also referred to as 3D laser scanning. In an example of use of such a sensor, a fixed or mobile device (such as a smart phone) having a LiDAR sensor can measure and map an area extending 5 meters or more away from the sensor. The LiDAR data can be fused with point cloud data estimated by an electromagnetic RADAR sensor, for example. The LiDAR sensor(s) can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of a user's face, a user interface 100 (e.g., when worn on a user's face), and/or an environment. In a further use, for solid surfaces through which radio waves pass (e.g., radio-translucent materials), the LiDAR may reflect off such surfaces, thus allowing a classification of different type of obstacles. While a LiDAR sensor is described herein, in some cases one or more other ranging sensors can be used instead of or in addition to a LiDAR sensor, such as an ultrasonic ranging sensor, an electromagnetic RADAR sensor, and the like.

Aside from being located on the RPT device 40, conduit or interface 100, any or all of the above describe sensors may be located on external devices such as a mobile user device or an activity tracker. For example, the audio sensor 4278 and speaker 4334 may integrated in and/or coupled to a mobile device and the pressure sensor 4272 and/or flow rate sensor 4274 are integrated in and/or coupled to the RPT device 40. In some implementations, at least one of the one or more sensors may be positioned generally adjacent to the user 10 during the sleep session (e.g., positioned on or in contact with a portion of the user 10, worn by the user 10, coupled to or positioned on the nightstand, coupled to the mattress, coupled to the ceiling, etc.).

In one form of the present technology, an RPT device 40 includes one or more input devices 4220 in the form of buttons, switches or dials to allow a person to interact with the device. The buttons, switches or dials may be physical devices, or software devices accessible via a touch screen. The buttons, switches or dials may, in one form, be physically connected to the external housing 4010, or may, in another form, be in wireless communication with a receiver that is in electrical connection to the central controller 4230. In one form, the input device 4220 may be constructed and arranged to allow a person to select a value and/or a menu option.

In one form of the present technology, the central controller 4230 is one or a plurality of processors suitable to control an RPT device 40. Suitable processors may include an x86 INTEL processor, a processor based on ARM® Cortex®-M processor from ARM Holdings such as an STM32 series microcontroller from ST MICROELECTRONIC. In certain alternative forms of the present technology, a 32-bit RISC CPU, such as an STR9 series microcontroller from ST MICROELECTRONICS or a 16-bit RISC CPU such as a processor from the MSP430 family of microcontrollers, manufactured by TEXAS INSTRUMENTS may also be suitable. In one form of the present technology, the central controller 4230 is a dedicated electronic circuit. In one form, the central controller 4230 is an application-specific integrated circuit. In another form, the central controller 4230 comprises discrete electronic components. The central controller 4230 may be configured to receive input signal(s) from one or more transducers 4270, one or more input devices 4220, and the humidifier 60.

The central controller 4230 may be configured to provide output signal(s) to one or more of an output device 4290, a therapy device controller 4240, a data communication interface 4280, and the humidifier 60.

In some forms of the present technology, the central controller 4230 is configured to implement the one or more methodologies described herein, such as the one or more algorithms expressed as computer programs stored in a non-transitory computer readable storage medium, on an internal memory. In some forms of the present technology, the central controller 4230 may be integrated with an RPT device 40. However, in some forms of the present technology, some methodologies may be performed by a remotely located device such as a mobile computing device. For example, the remotely located device may determine control settings for a ventilator or detect respiratory related events by analysis of stored data such as from any of the sensors described herein. As explained above, all data and operations for external sources or the central controller 4230 are generally proprietary to the manufacturer of the RPT device 40. Thus, the data from the sensors and any other additional operational data is not generally accessible by any other device.

In one form of the present technology, a data communication interface is provided, and is connected to the central controller 4230. The data communication interface may be connectable to a remote external communication network and/or a local external communication network. The remote external communication network may be connectable to remote external devices such as servers or databases. The local external communication network may be connectable to a local external device such as a mobile device or a health monitoring device. Thus, the local external communication network may be used by either the RPT device 40 or a mobile device to collect data from other devices.

In one form, the data communication interface is part of the central controller 4230. In another form, data communication interface 4280 is separate from the central controller 4230, and may comprise an integrated circuit or a processor. In one form, the remote external communication network is the Internet. The data communication interface may use wired communication (e.g. via Ethernet, or optical fiber) or a wireless protocol (e.g. CDMA, GSM, 2G, 3G, 4G/LTE, LTE Cat-M, NB-IoT, 5G New Radio, satellite, beyond 5G) to connect to the Internet. In one form, local external communication network 4284 utilizes one or more communication standards, such as Bluetooth, or a consumer infrared protocol.

The example RPT device 40 includes integrated sensors and communication electronics as shown in FIG. 4C. Older RPT devices may be retrofitted with a sensor module that may include communication electronics for transmitting collected data. Such a sensor module could be attached to the RPT device and thus transmit operational data to a remote analysis engine in the server 210.

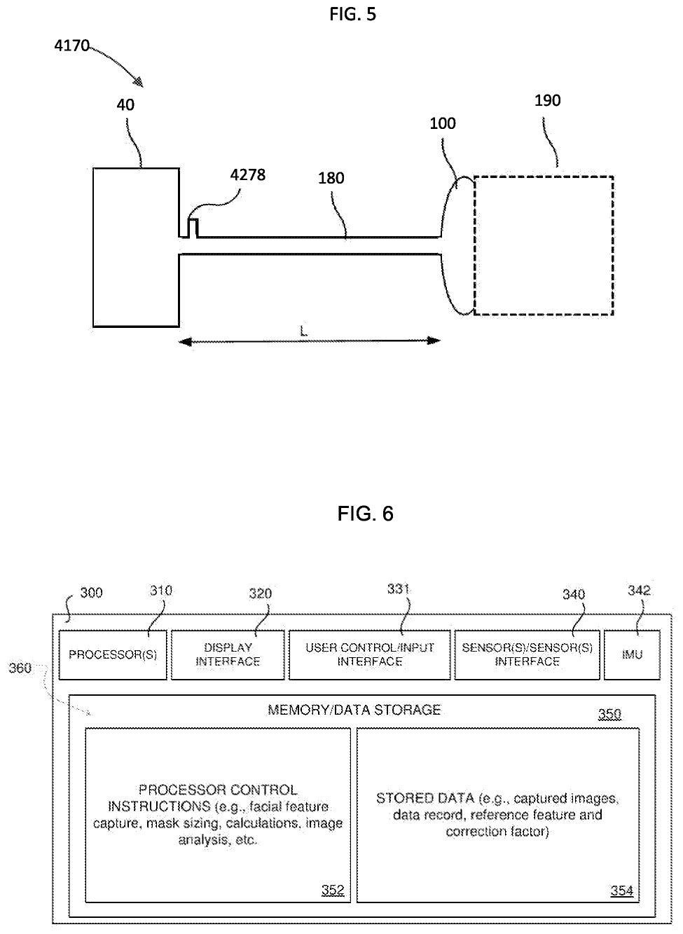

FIG. 5 shows a diagram of the audio sensor 4278 in the air circuit 4170 joining the RPT device 40 to the interface 100 in FIG. 1. In this example a conduit 180 (of length L) effectively acts as an acoustic waveguide for sound produced by the RPT device 40. In this example the input signal is sound emitted by the RPT device 40. The input signal (e.g., an impulse) enters the audio sensor 4278 positioned at one end of the conduit 180, travels along the airpath in conduit 180 to mask 100 and is reflected back along the conduit 180 by features in the airpath (which includes the conduit 180 and the mask 100) to enter the audio sensor 4278 once more. The system IRF (the output signal produced by an input impulse) therefore contains an input signal component and a reflection component. A key feature is the time taken by sound to travel from one end of the airpath to the opposite end. This interval is manifested in the system IRF because the audio sensor 4278 receives the input signal coming from the RPT device 40, and then some time later receives the input signal filtered by the conduit 180 and reflected and filtered by the mask 100 (and potentially any other system 190 attached to the mask, e.g. a human respiratory system when the mask 100 is seated on a user). This means that the component of the system IRF associated with the reflection from the mask end of the conduit 180 (the reflection component) is delayed in relation to the component of the system IRF associated with the input signal (the input signal component), which arrives at the audio sensor 4278 after a relatively brief delay. (For practical purposes, this brief delay may be ignored and zero time approximated to be when the microphone first responds to the input signal.) The delay is equal to 2 L/c (wherein L is the length of the conduit, and c is the speed of sound in the conduit).

Another feature is that because the airpath is loss-prone, provided the conduit is long enough, the input signal component decays to a negligible amount by the time the reflection component of the system IRF has begun. If this is the case, then the input signal component may be separated from the reflection component of the system IRF. Alternatively, the input signal may originate from a speaker at the device end of the airpath

Some implementations of the disclosed acoustic analysis technologies may implement cepstrum analysis. A cepstrum may be considered the inverse Fourier Transform of the log spectrum of the forward Fourier Transform of the decibel spectrum, etc. The operation essentially can convert a convolution of an impulse response function (IRF) and a sound source into an addition operation so that the sound source may then be more easily accounted for or removed so as to isolate data of the IRF for analysis. Techniques of cepstrum analysis are described in detail in a scientific paper entitled “The Cepstrum: A Guide to Processing” (Childers et al, Proceedings of the IEEE, Vol. 65, No. 10, October 1977) and Randall RB, Frequency Analysis, Copenhagen: Bruel & Kjaer, p. 344 (1977, revised ed. 1987). The application of cepstrum analysis to respiratory therapy system component identification is described in detail in PCT Publication No. WO2010/091462, titled “Acoustic Detection for Respiratory Treatment Apparatus,” the entire contents of which are hereby incorporated by reference.

As previously mentioned, a respiratory therapy system typically includes a respiratory therapy device (e.g., an RPT device), a humidifier, an air delivery conduit, and a patient interface such as those components shown in FIG. 1. A variety of different forms of patient interfaces may be used with a given RPT device, for example nasal pillows, nasal prongs, nasal masks sealing over the nose ridge, nasal-only masks sealing at an inferior periphery of the nose instead of over the nose ridge, nose & mouth (oronasal) masks sealing either over the nose bridge in the manner of a traditional full-face mask or sealing at an inferior periphery of the nose instead of over the nose ridge, tube-down masks in which a conduit connects to an anterior-facing portion of the mask, masks having conduit headgear, masks having an integrated short tube to which the main conduit is connected, and masks which have a decoupling structure such as an elbow to which the main conduit is directly connected among other types of masks and variations of the above. Furthermore, different forms of air delivery conduits may be used. In order to provide improved control of therapy delivered to the user interface, measuring or estimating treatment parameters such as pressure in the user interface, and vent flow may be analyzed. In older systems knowledge of the type of component being used by a user can be determined as will be explained below to determine optimal interfaces for a user. Some RPT devices include a menu system that allows the user to select the type of system components, including the user interface, being used, e.g., brand, form, model, etc. Once the types of the components are entered by the user, the RPT device can select appropriate operating parameters of the flow generator that best coordinate with the selected components. The data collected by the RPT device may be used to evaluate the effectiveness of the particular selected components such as a user interface in supplying pressurized air to the user.

Acoustic analysis may be used to identify components of the respiratory pressure therapy system as explained above in reference to FIG. 5. In the present specification, “identification” of a component means identification of the type of that component. In what follows, “mask” is used synonymously with “user interface” for brevity, even though there exist user interfaces that are not usually described as “masks.”

The system may identify the length of the conduit in use, as well as the mask connected to the conduit via analysis of a sound signal acquired by the audio sensor 4278. The technology may identify the mask and conduit regardless of whether a user is wearing the mask at the time of identification.

This technology includes an analysis method that enables the separation of the acoustic mask reflections from the other system noises and responses, including but not limited to blower sound. This makes it possible to identify differences between acoustic reflections (usually dictated by mask shapes, configurations and materials) from different masks and may permit the identification of different masks without user or user intervention.

An example method of identifying the mask is to sample the output sound signal y(t) acquired by the audio sensor 4278 at at least the Nyquist rate, for example 20 kHz, compute the cepstrum from the sampled output signal, and then separate a reflection component of the cepstrum from the input signal component of the cepstrum. The reflection component of the cepstrum comprises the acoustic reflection from the mask of the input sound signal, and is therefore referred to as the “acoustic signature” of the mask, or the “mask signature.” The acoustic signature is then compared with a predefined or predetermined database of previously measured acoustic signatures obtained from systems containing known masks. Optionally, some criteria would be set to determine appropriate similarity. In one example embodiment, the comparisons may be completed based on the single largest data peak in the cross-correlation between the measured and stored acoustic signatures. However, this approach may be improved by comparisons over several data peaks or alternatively, wherein the comparisons are completed on extracted unique sets of cepstrum features.

Alternatively, the same method may be also used to determine the conduit length, by finding the delay between a sound being received from the RPT device 40 and its reflection from the mask 100 being received; the delay may be proportional to the length of the tube 180. Additionally, changes in tubing diameter may increase or decrease the amplitude of the reflected signals and therefore may also be identifiable. Such an assessment may be made by comparison of current reflection data with prior reflection data. The diameter change may be considered as a proportion of the change in amplitude from the reflected signals (i.e., reflection data).

In accordance with the present technology, data associated with the reflection component, may then be compared with similar data from previously identified mask reflection components such as that contained in a memory or database of mask reflection components.

For example, the reflection component of a mask being tested (the “mask signature”) may be separated from the cepstrum of the output signal generated by the microphone. This mask signature may be compared to that of previous or predetermined mask signatures of known masks stored as a data template of the apparatus. One way of doing this is to calculate the cross-correlation between the mask signature of the mask being tested and previously stored mask signatures for all known masks or data templates. There is a high probability that the cross-correlation with the highest peak corresponds to the mask being tested, and the location of the peak should be proportional to the length of the conduit.

As explained above, the RPT device 40 may provide data for the type of user interface as well as operational data. The operational data may be correlated with the mask type and data relating to the user to determine whether a particular mask type is effective. For example, the operational data reflects both the use times of the RPT device 40 as well as whether the use provides effective therapy. Types of user interfaces may be correlated with the level of user compliance or effectiveness of therapy as determined from the operational data collected by the RPT device 40. The correlated data may be used to better determine an effective interface for new users requiring respiratory therapy from a similar RPT device. This selection may be combined with facial dimensions obtained from a scan of the face of the new user to assist in the selection of an interface.

Thus, example of the present technology may allow users to more quickly and conveniently obtain a user interface such as a mask by integrating data gathered from use of RPT devices in relation to different masks by a user population with facial features of the individual user determined by a scanning process. The scanning process allows a user to quickly measure their facial anatomy from the comfort of their own home using a computing device, such as a desktop computer, tablet, smart phone or other mobile device. The computing device may then receive or generate a recommendation for an appropriate user interface size and type after analysis of the facial dimensions of the user and data from a general user population using a variety of different interfaces.

In a beneficial embodiment, the present technology may employ an application downloadable from a manufacturer or third-party server to a smart phone or tablet with an integrated camera. When launched, the application may provide visual and/or audio instructions. As instructed, the user (i.e., a user) may stand in front of a mirror, and press the camera button on a user interface. An activated process may then take a series of pictures of the user's face, and then, within a matter of seconds for example, obtain facial dimensions for selection of an interface (based on the processor analyzing the pictures).

A user/user may capture an image or series of images of their facial structure. One example may be instructions provided by an application stored on a computer-readable medium, such as when executed by a processor, detect various facial landmarks within the images, measure and scale the distance between such landmarks, compare these distances to a data record, and recommend an appropriate user interface size. Thus, an automated device of a consumer may permit accurate user interface selection, such as in the home, to permit customers to determine sizing without trained associates.

Other examples may include identification of three-dimensional facial features from the images. Identification of facial features is sizing based on the “shape” of different features. The shape is described as the near continuous surface of a user's face. In reality, a continuous surface is not possible, but collecting around 10 k-100 k points on the face provides an approximation of the continuous surface of the face. There are several example techniques for collecting facial image data for identifying three-dimensional facial features.

One method may be determining the facial images from a 2D image. In this method, computer vision (CV) and a trained machine learning (ML) model are employed to extract key facial landmarks. For example, OpenCV and DLib libraries may be used for landmark comparison through having a trained number of standard facial landmarks. Once the preliminary facial landmarks are extracted, the derived three-dimensional features must be properly scaled. Scaling involves determining an object such as a coin, credit card or the iris of the user to provide a known scale. For example, Google Mediapipe Facemesh and Iris models may track the iris of a user and scale face landmarks for the purposes of mask sizing. These models contain 468 landmarks of the face and 10 landmarks of the eyes. The iris data is then used to scale other identified facial features.

Another method of determining three-dimensional features may be from facial data taken from a 3D camera with a depth sensor. 3D cameras (such as that on the iPhone X and above) can perform a 3D scan of a face and return a meshed (triangulated) surface. The number of surface points is generally in the order of ˜50 k. In this example, there are 2 types of outputs from a 3D camera such as the iPhone. These are: (a) raw scan data, and (b) a lower resolution blendshape model used for face detection and tracking. The latter includes automatic landmarking, whereas the former does not. The mesh surface data does not require scaling.

Another method is generating a 3D model directly from a 2D image. This involves using a 3D morphable model (or 3DMM) and machine learning to adapt the shape of the 3DMM to match the face in the image. Single or multiple image views are possible from multiple angles and may be derived from a video captured on a digital camera. The 3DMM may be adapted to match the data taken from the multiple 2D images via a machine learning matching routine. The 3DMM may be adapted to account for the shape, pose, and expression shown in the facial image to modify the facial features. Scaling may still be required, and thus detection and scaling of a known object such as an eye feature such as an iris could be used as a reference to account for scaling errors due to factors such as age.

The three-dimensional features or shape data may be used for mask sizing. One way to match a mask is aligning the identified surfaces of the face with the known surfaces of the proposed mask. The surfaces are then aligned. The alignment may be performed by the nearest iterative closest point (NICP) technique. A fit score may then be calculated by determining the mean average distances, which is the mean of distances between the closest or corresponding points of the facial features and the mask contact surfaces. A low score corresponds to a good fit.

Another method of mask sizing may be to use 3D face scans collected from different users. In this example, 3D data may be collected for over 1,000 users. These users are grouped according to their ideal mask size. In this example, the number of ideal mask sizes available are determined by mask designers for covering different user types. This method of grouping can be grouped based on other types of data, such as grouping according to traditional 2D landmarks or grouping on principal components of face shape. The principal component analysis may be used for determining a reduced set of characteristics of facial features. An average set of 3D facial features that represent each mask size is calculated based on the groupings of mask sizes.

To size a new user, a 3D facial scan is taken or 3D data is derived from 2D images, and the fit score for the new user is calculated to each of the average faces. The mask size and type of mask selected is the mask corresponding to the average face with the lowest fit score. Additional personal preferences may be incorporated. The specific facial features could also be used to create a customized sizing based on modifying one of the available mask types.

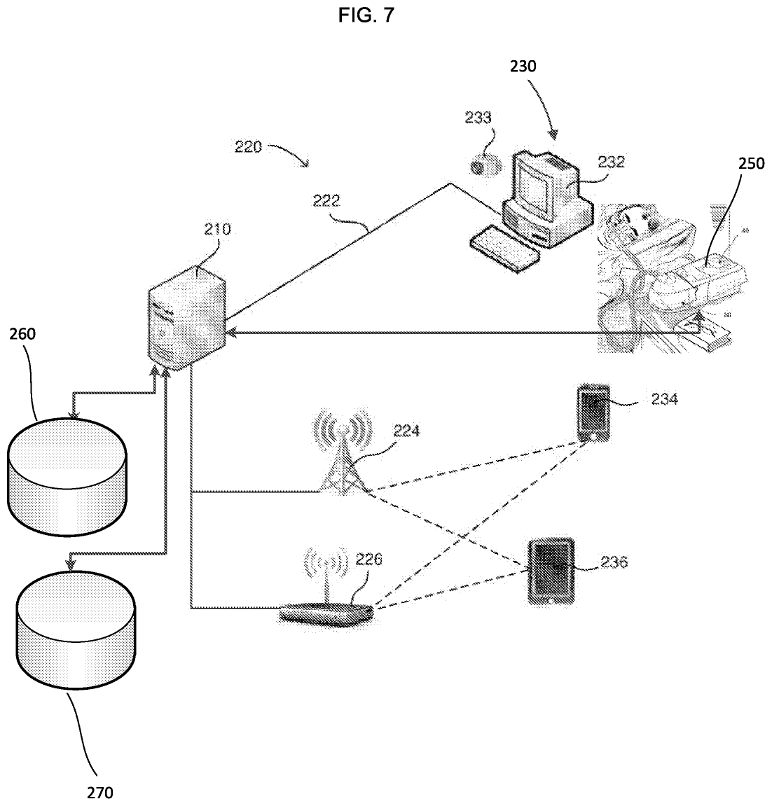

FIG. 7 depicts an example system 200 that may be implemented for automatic facial feature measuring and user interface selection. System 200 may generally include one or more of servers 210, a communication network 220, and a computing device 230. Server 210 and computing device 230 may communicate via communication network 220, which may be a wired network 222, wireless network 224, or wired network with a wireless link 226. In some versions, server 210 may communicate one-way with computing device 230 by providing information to computing device 230, or vice versa. In other embodiments, server 210 and computing device 230 may share information and/or processing tasks. The system may be implemented, for example, to permit automated purchase of user interfaces such as the mask 100 in FIG. 1 where the process may include automatic sizing processes described in more detail herein. For example, a customer may order a mask online after running a mask selection process that automatically identifies a suitable mask size, type and/or model by image analysis of the customer's facial features in combination with operational data from other masks and RPT device operational data from a patient population using different types and sizes of masks. The system 200 may comprise one or more databases. The one or more databases may include a patient database 260, patient interface database 270 and any other database described herein. It is to be understood that in some examples of the present technology, all data required to be accessed by a system or during performance of a method may be stored in a single database. In other examples the data may be stored in two or more separate databases. Accordingly, where there is a reference herein to a particular database, it is to be understood that in some examples the particular database may be a distinct database and in other examples it may be part of a larger database.

The server 210 and or the computing device 230 may also be in communication with a respiratory therapy device such as an RPT device 250 similar to the RPT device 40 shown in FIG. 1. The RPT device 250 in this example collects operational data in relation to user use, mask leaks, and other relevant data to provide feedback in relation to mask use. The data from the RPT devices 250 is collected and correlated with the individual user data of the user using the RPT devices 250 in a user database 260. A user interface database 270 includes data on different types and sizes of interfaces such as masks that may be available for a new user. The user interface database 270 may also include acoustic signature data of each type of mask that may enable the determination of mask type from audio data collected from respiratory therapy devices. A mask analysis engine executed by the server 210 is used to correlate and determine effective mask sizes and types from the individual facial dimensional data and corresponding effectiveness from operational data collected by the RPT devices 250 encompassing an entire user population. For example, an effective fit may be evidenced by minimum detected leaks, maximum compliance with a therapy plan (e.g., mask on and off times, frequency of on and off events, therapy pressure used), number of apneas overnight, AHI levels, pressure settings used on their device and also prescribed pressure settings. This data may be correlated with facial dimensional data or other data based on the facial image of a new user.

For example, the face shape derived from imaging the face of the user such as 3D scan data) may be compared to the geometry of the features of a proposed mask (cushion, conduit, headgear). The difference between the shape and the geometry may be analyzed to determine if there are fit issues that might result in leaks or high contact pressure regions leading to redness/soreness from the contact areas. As explained herein, the data gathered for a population of users, may be combined with other forms of data such as detected leaks to identify the best mask system for a particular face shape (i.e. shape of mouth, nose, cheeks, head etc.).

As will be explained, the server 210 collects the data from multiple users stored in the database 260 and corresponding mask size and type data stored in the database 270 to select an appropriate mask based on the optimal mask that best fits the scanned facial dimensional data collected from the new user and the masks that achieved the best operational data for users that have facial dimensions and other features, sleep behavioral data, and demographic data that are similar to the new user. In some examples, the system 200 comprises one or more databases for storing a plurality of facial features from a user population and a corresponding plurality of user interfaces used by the user population, and operational data of respiratory pressure therapy devices used by the user population with the plurality of corresponding user interfaces. A selection engine may be coupled to the one or more databases, the selection engine being operable to select a user interface for the user based on a desired outcome and based on the stored operational data and facial features of the user. The system 200 may be configured to perform a corresponding method of selecting a user interface. Thus, the system 200 may provide a mask recommendation to a new user by determining what mask has been shown to be optimal for existing users similar in various ways to the new user. The optimal mask may be the mask type, model and/or size that has been shown to be associated with greatest compliance with therapy, lowest leak, fewest apneas, lowest AHI and most positive subjective user feedback, for example. The influence of each of these results in the determination of the optimal mask may be given various different weightings in various examples of the present technology.

The computing device 230 can be a desktop or laptop computer 232 or a mobile device, such as a smart phone 234 or tablet 236. FIG. 7 depicts the general architecture 300 of the computing device 230. Device 230 may include one or more processors 310. Device 230 may also include a display interface 320, user control/input interface 331, sensor 340 and/or a sensor interface for one or more sensor(s), inertial measurement unit (IMU) 342 and non-volatile memory/data storage 350.

Sensor 340 may be one or more cameras (e.g., a CCD charge-coupled device or active pixel sensors) that are integrated into computing device 230, such as those provided in a smart phone or in a laptop. Alternatively, where computing device 230 is a desktop computer, device 230 may include a sensor interface for coupling with an external camera, such as the webcam 233 depicted in FIG. 6. Other exemplary sensors that could be used to assist in the methods described herein that may either be integral with or external to the computing device include stereoscopic cameras, for capturing three-dimensional images, or a light detector capable of detecting reflected light from a laser or strobing/structured light source.

User control/input interface 331 allows the user to provide commands or respond to prompts or instructions provided to the user. This could be a touch panel, keyboard, mouse, microphone, and/or speaker, for example.

Display interface 320 may include a monitor, LCD panel, or the like to display prompts, output information (such as facial measurements or interface size recommendations), and other information, such as a capture display, as described in further detail below.

Memory/data storage 350 may be the computing device's internal memory, such as RAM, flash memory or ROM. In some embodiments, memory/data storage 350 may also be external memory linked to computing device 230, such as an SD card, server, USB flash drive or optical disc, for example. In other embodiments, memory/data storage 350 can be a combination of external and internal memory. Memory/data storage 350 includes stored data 354 and processor control instructions 352 that instruct processor 310 to perform certain tasks. Stored data 354 can include data received by sensor 340, such as a captured image, and other data that is provided as a component part of an application. Processor control instructions 352 can also be provided as a component part of an application.

As explained above, a facial image may be captured by a mobile computing device such as the smart phone 234. An appropriate application executed on the computing device 230 or the server 210 can provide three-dimensional relevant facial data to assist in selection of an appropriate mask. The application may use any appropriate method of facial scanning Such applications may include the Capture from StandardCyborg (https://www.standardcyborg.com/), an application from Scandy Pro (https://www.scandy.co/products/scandy-pro), the Beauty 3D application from Qianxun3d (http://www.gianxun3d.com/scanpage), the Unre 3D FaceApp (http://www.unre.ai/index.php?route=ios/detail) and an application from Bellus 3D (https://www.bellus3d.com/) A detailed process of facial scanning include the techniques disclosed in WO 2017000031, hereby incorporated by reference in its entirety.

One such application is an application for facial feature measuring and/or user interface sizing 360, which may be an application downloadable to a mobile device, such as smart phone 234 and/or tablet 236. The application 360, which may be stored on a computer-readable medium, such as memory/data storage 350, includes programmed instructions for processor 310 to perform certain tasks related to facial feature measuring and/or user interface sizing. The application also includes data that may be processed by the algorithm of the automated methodology. Such data may include a data record, reference feature, and correction factors, as explained in additional detail below.

The application 360 is executed by the processor 310, to measure user facial features using two-dimensional or three-dimensional images and to select appropriate user interface sizes and types, such as from a group of standard sizes, based on the resultant measurements. The method may generally be characterized as including three or four different phases: a pre-capture phase, a capture phase, a post-capture image processing phase, and a comparison and output phase.

In some cases, the application for facial feature measuring and user interface sizing may control a processor 310 to output a visual display that includes a reference feature on the display interface 320. The user may position the feature adjacent to their facial features, such as by movement of the camera. The processor may then capture and store one or more images of the facial features in association with the reference feature when certain conditions, such as alignment conditions are satisfied. This may be done with the assistance of a mirror. The mirror reflects the displayed reference feature and the user's face to the camera. The application then controls the processor 310 to identify certain facial features within the images and measure distances therebetween. By image analysis processing a scaling factor may then be used to convert the facial feature measurements, which may be pixel counts, to standard mask measurement values based on the reference feature. Such values may be, for example, standardized unit of measure, such as a meter or an inch, and values expressed in such units suitable for mask sizing.

Additional correction factors may be applied to the measurements. The facial feature measurements may be compared to data records that include measurement ranges corresponding to different user interface sizes for particular user interface forms, such as nasal masks and FFMs, for example. The recommended size may then be chosen and be output to the user/patient based on the comparison(s) as a recommendation. Such a process may be conveniently effected within the comfort of any preferred user location. The application may perform this method within seconds. In one example, the application performs this method in real time.

In the pre-capture phase, the processor 310, among other things, assists the user in establishing the proper conditions for capturing one or more images for sizing processing. Some of these conditions include proper lighting and camera orientation and motion blur caused by an unsteady hand holding the computing device 230, for example.

A user may conveniently download an application for performing the automatic measuring and sizing at a user device such as a computing device 230 from a server, such as a third party application-store server, onto their computing device 230. When downloaded, such application may be stored on the computing device's internal non-volatile memory, such as RAM or flash memory. Computing device 230 is preferably a mobile device, such as smart phone 234 or tablet 236.

When the user launches the application, processor 310 may prompt the user via the display interface 320 to provide user specific information, such as age, gender, weight, and height. However, processor 310 may prompt to the user to input this information at any time, such as after the user's facial features are measured. Processor 310 may also present a tutorial, which may be presented audibly and/or visually, as provided by the application to aid the user in understanding their role during the process. The prompts may also require information for user interface type, e.g., nasal or full face, etc. and of the type of device for which the user interface will be used. Also, in the pre-capture phase, the application may extrapolate the user specific information based on information already gathered by the user, such as after receiving captured images of the user's face, and based on machine learning techniques or through artificial intelligence.

When the user is prepared to proceed, which may be indicated by a user input or response to a prompt via user control/input interface 331, processor 310 activates sensor 340 as instructed by the application's processor control instructions 352. Sensor 340 is preferably the mobile device's forward facing camera, which is located on the same side of the mobile device as display interface 320. The camera is generally configured to capture two-dimensional images. Mobile device cameras that capture two-dimensional images are ubiquitous. The present technology takes advantage of this ubiquity to avoid burdening the user with the need to obtain specialized equipment.

Around the same time sensor/camera 340 is activated, processor 310, as instructed by the application, presents a capture display on the display interface 320. The capture display may include a camera live action preview, a reference feature, a targeting box, and one or more status indicators or any combination thereof. In this example, the reference feature is displayed centered on the display interface and has a width corresponding to the width of the display interface 320. The vertical position of the reference feature may be such that the top edge of reference feature abuts the upper most edge of the display interface 320 or the bottom edge of reference feature abuts the lower most edge of the display interface 320. A portion of the display interface 320 will display the camera live action preview 324, typically showing the user's facial features captured by sensor/camera 340 in real time if the user is in the correct position and orientation.

The reference feature is a feature that is known to computing device 230 (predetermined) and provides a frame of reference to processor 310 that allows processor 310 to scale captured images. The reference feature may preferably be a feature other than a facial or anatomical feature of the user. Thus, during the image processing phase, the reference feature assists processor 310 in determining when certain alignment conditions are satisfied, such as during the pre-capture phase. The reference features may be a quick response (QR) code or known exemplar or marker, which can provide processor 310 certain information, such as scaling information, orientation, and/or any other desired information which can optionally be determined from the structure of the QR code. The QR code may have a square or rectangular shape. When displayed on display interface 320, the reference feature has predetermined dimensions, such as in units of millimeters or centimeters, the values of which may be coded into the application and communicated to processor 310 at the appropriate time. The actual dimensions of reference feature 326 may vary between various computing devices. In some versions, the application may be configured to be a computing device model-specific in which the dimensions of reference feature 326, when displayed on the particular model, is already known. However, in other embodiments, the application may instruct processor 310 to obtain certain information from device 230, such as display size and/or zoom characteristics that allow the processor 310 to compute the real world/actual dimensions of the reference feature as displayed on display interface 320 via scaling. Regardless, the actual dimensions of the reference feature as displayed on the display interfaces 320 of such computing devices are generally known prior to post-capture image processing.

Along with the reference feature, the targeting box may be displayed on display interface 320. The targeting box allows the user to align certain components within capture display 322 in targeting box, which is desired for successful image capture.

The status indicator provides information to the user regarding the status of the process. This helps ensure the user does not make major adjustments to the positioning of the sensor/camera prior to completion of image capture.

Thus, when the user holds display interface 320 parallel to the facial features to be measured and presents user display interface 320 to a mirror or other reflective surface, the reference feature is prominently displayed and overlays the real-time images seen by camera/sensor 340 and as reflected by the mirror. This reference feature may be fixed near the top of display interface 320. The reference feature is prominently displayed in this manner at least partially so that sensor 340 can clearly see the reference feature so that processor 310 can easily the identify feature. In addition, the reference feature may overlay the live view of the user's face, which helps avoid user confusion.

The user may also be instructed by processor 310, via display interface 320, by audible instructions via a speaker of the computing device 230, or be instructed ahead of time by the tutorial, to position display interface 320 in a plane of the facial features to be measured. For example, the user may be instructed to position display interface 320 such that it is facing anteriorly and placed under, against, or adjacent to the user's chin in a plane aligned with certain facial features to be measured. For example, display interface 320 may be placed in planar alignment with the sellion and suprementon. As the images ultimately captured are two-dimensional, planar alignment helps ensure that the scale of reference feature 326 is equally applicable to the facial feature measurements. In this regard, the distance between the mirror and both of the user's facial features and the display will be approximately the same.

When the user is positioned in front of a mirror and display interface 320, which includes the reference feature, is roughly placed in planar alignment with the facial features to be measured, processor 310 checks for certain conditions to help ensure sufficient alignment. One exemplary condition that may be established by the application, as previously mentioned, is that the entirety of the reference feature must be detected within targeting box 328 in order to proceed. If processor 310 detects that the reference feature is not entirely positioned within targeting box, the processor 310 may prohibit or delay image capture. The user may then move their face along with display interface 320 to maintain planarity until the reference feature, as displayed in the live action preview, is located within targeting box. This helps optimized alignment of the facial features and display interface 320 with respect to the mirror for image capture.

When processor 310 detects the entirety of reference feature within targeting box, processor 310 may read the IMU 342 of the computing device for detection of device tilt angle. The IMU 342 may include an accelerometer or gyroscope, for example. Thus, the processor 310 may evaluate device tilt such as by comparison against one or more thresholds to ensure it is in a suitable range. For example, if it is determined that computing device 230, and consequently display interface 320 and user's facial features, is tilted in any direction within about ±5 degrees, the process may proceed to the capture phase. In other embodiments, the tilt angle for continuing may be within about ±10 degrees, ±7 degrees, ±3 degrees, or ±1 degree. If excessive tilt is detected a warning message may be displayed or sounded to correct the undesired tilt. This is particularly useful for assisting the user to help prohibit or reduce excessive tilt, particularly in the anterior-posterior direction, which if not corrected, could pose as a source of measuring error as the captive reference image will not have a proper aspect ratio.

When alignment has been determined by processor 310 as controlled by the application, processor 310 proceeds into the capture phase. The capture phase preferably occurs automatically once the alignment parameters and any other conditions precedent are satisfied. However, in some embodiments, the user may initiate the capture in response to a prompt to do so.

When image capture is initiated, the processor 310 via sensor 340 captures a number n of images, which is preferably more than one image. For example, the processor 310 via sensor 340 may capture about 5 to 20 images, 10 to 20 images, or 10 to 15 images, etc. The quantity of images captured may be time-based. In other words, the number of images that are captured may be based on the number of images of a predetermined resolution that can be captured by sensor 340 during a predetermined time interval. For example, if the number of images sensor 340 can capture at the predetermined resolution in 1 second is 40 images and the predetermined time interval for capture is 1 second, sensor 340 will capture 40 images for processing with processor 310. The quantity of images may be user-defined, determined by server 210 based on artificial intelligence or machine learning of environmental conditions detected, or based on an intended accuracy target. For example, if high accuracy is required then more captured images may be required. Although, it is preferable to capture multiple images for processing, one image is contemplated and may be successful for use in obtaining accurate measurements. However, more than one image allows average measurements to be obtained. This may reduce error/inconsistencies and increase accuracy. The images may be placed by processor 310 in stored data 354 of memory/data storage 350 for post-capture processing.

Once the images are captured, the images are processed by processor 310 to detect or identify facial features/landmarks and measure distances between landmarks. The resultant measurements may be used to recommend an appropriate user interface size. This processing may alternatively be performed by server 210 receiving the transmitted captured images and/or on the user's computing device (e.g., smart phone). Processing may also be undertaken by a combination of the processor 310 and server 210. In one example, the recommended user interface size may be predominantly based on the user's nose width. In other examples, the recommended user interface size may be based on the user's mouth and/or nose dimensions.

Processor 310, as controlled by the application, retrieves one or more captured images from stored data 354. The image is then extracted by processor 310 to identify each pixel comprising the two-dimensional captured image. Processor 310 then detects certain pre-designated facial features within the pixel formation.

Detection may be performed by processor 310 using edge detection, such as Canny, Prewitt, Sobel, or Robert's edge detection, for example. These edge detection techniques/algorithms help identify the location of certain facial features within the pixel formation, which correspond to the user's actual facial features as presented for image capture. For example, the edge detection techniques can first identify the user's face within the image and also identify pixel locations within the image corresponding to specific facial features, such as each eye and borders thereof, the mouth and corners thereof, left and right alares, sellion, supramenton, glabella and left and right nasolabial sulci, etc. Processor 310 may then mark, tag or store the particular pixel location(s) of each of these facial features. Alternatively, or if such detection by processor 310/server 210 is unsuccessful, the pre-designated facial features may be manually detected and marked, tagged or stored by a human operator with viewing access to the captured images through a user interface of the processor 310/server 210.

Once the pixel coordinates for these facial features are identified, the application controls processor 310 to measure the pixel distance between certain of the identified features. For example, the distance may generally be determined by the number of pixels for each feature and may include scaling. For example, measurements between the left and right alares may be taken to determine pixel width of the nose and/or between the sellion and supramenton to determine the pixel height of the face. Other examples include pixel distance between each eye, between mouth corners, and between left and right nasolabial sulci to obtain additional measurement data of particular structures like the mouth. Further distances between facial features can be measured. In this example, certain facial dimensions are used for the user interface selection process.

Once the pixel measurements of the pre-designated facial features are obtained, an anthropometric correction factor(s) may be applied to the measurements. It should be understood that this correction factor can be applied before or after applying a scaling factor, as described below. The anthropometric correction factor can correct for errors that may occur in the automated process, which may be observed to occur consistently from user to user. In other words, without the correction factor, the automated process, alone, may result in consistent results from user to user, but results that may lead to a certain amount of mis-sized user interfaces. The correction factor, which may be empirically extracted from population testing, shifts the results closer to a true measurement helping to reduce or eliminate mis-sizing. This correction factor can be refined or improved in accuracy over time as measurement and sizing data for each user is communicated from respective computing devices to server 210 where such data may be further processed to improve the correction factor. The anthropometric correction factor may also vary between the forms of user interfaces. For instance, the correction factor for a particular user seeking an FFM may be different from the correction factor when seeking a nasal mask. Such a correction factor may be derived from tracking of mask purchases, such as by monitoring mask returns and determining the size difference between a replacement mask and the returned mask.

In order to apply the facial feature measurements to user interface sizing, whether corrected or uncorrected by the anthropometric correction factor, the measurements may be scaled from pixel units to other values that accurately reflect the distances between the user's facial features as presented for image capture. The reference feature may be used to obtain a scaling value or values. Thus, processor 310 similarly determines the reference feature's dimensions, which can include pixel width and/or pixel height (x and y) measurements (e.g., pixel counts) of the entire reference feature. More detailed measurements of the pixel dimensions of the many squares/dots that comprise a QR code reference feature, and/or pixel area occupied by the reference feature and its constituent parts may also be determined. Thus, each square or dot of the QR code reference feature may be measured in pixel units to determine a scaling factor based on the pixel measurement of each dot and then averaged among all the squares or dots that are measured, which can increase accuracy of the scaling factor as compared to a single measurement of the full size of the QR code reference feature. However, it should be understood that whatever measurements are taken of the reference feature, the measurements may be utilized to scale a pixel measurement of the reference feature to a corresponding known dimension of the reference feature.

Once the measurements of the reference feature are taken by processor 310, the scaling factor is calculated by processor 310 as controlled by the application. The pixel measurements of reference feature are related to the known corresponding dimensions of the reference feature, e.g., the reference feature 326 as displayed by display interface 320 for image capture, to obtain a conversion or scaling factor. Such a scaling factor may be in the form of length/pixel or area/pixelA2. In other words, the known dimension(s) may be divided by the corresponding pixel measurement(s) (e.g., count(s)).

Processor 310 then applies the scaling factor to the facial feature measurements (pixel counts) to convert the measurements from pixel units to other units to reflect distances between the user's actual facial features suitable for mask sizing. This may typically involve multiplying the scaling factor by the pixel counts of the distance(s) for facial features pertinent for mask sizing.

These measurement steps and calculation steps for both the facial features and reference feature are repeated for each captured image until each image in the set has facial feature measurements that are scaled and/or corrected.

The corrected and scaled measurements for the set of images may then optionally be averaged by processor 310 to obtain final measurements of the user's facial anatomy. Such measurements may reflect distances between the user's facial features.

In the comparison and output phase, results from the post-capture image processing phase may be directly output (displayed) to a person of interest or compared to data record(s) to obtain an automatic recommendation for a user interface size.

Once all of the measurements are determined, the results (e.g., averages) may be displayed by processor 310 to the user via display interface 320. In one embodiment, this may end the automated process. The user/patient can record the measurements for further use by the user.

Alternatively, the final measurements may be forwarded either automatically or at the command of the user to server 210 from computing device 230 via communication network 220. Server 210 or individuals on the server-side may conduct further processing and analysis to determine a suitable user interface and user interface size.

In a further embodiment, the final facial feature measurements that reflect the distances between the actual facial features of the user are compared by processor 310 to user interface size data such as in a data record. The data record may be part of the application for automatic facial feature measurements and user interface sizing. This data record can include, for example, a lookup table accessible by processor 310, which may include user interface sizes corresponding to a range of facial feature distances/values. Multiple tables may be included in the data record, many of which may correspond to a particular form of user interface and/or a particular model of user interface offered by the manufacturer.

The example process for selection of user interfaces identifies key landmarks from the facial image captured by the above mentioned method. In this example, initial correlation to potential interfaces involves facial landmarks including face height, nose width and nose depth as represented by lines 3010, 3020 and 3030 in FIGS. 3A-3B. These three facial landmark measurements are collected by the application to assist in selecting the size of a compatible mask such as through the lookup table or tables described above. Alternatively, other data relating to facial 3D shapes may also be used for matching the derived shape data with the surfaces of the available masks as described above. For example, landmarks and any area of the face (i.e. mouth, nose etc.) can be obtained by fitting a 3D morphable model (3DMM) onto a 3D face scan of a user. This fitting process is also known as non-rigid registration or (shrink) wrapping. Once a 3DMM is registered to a 3D scan, the mask size may be determined using any number of methods, as the points and surface of the user's face are all known.

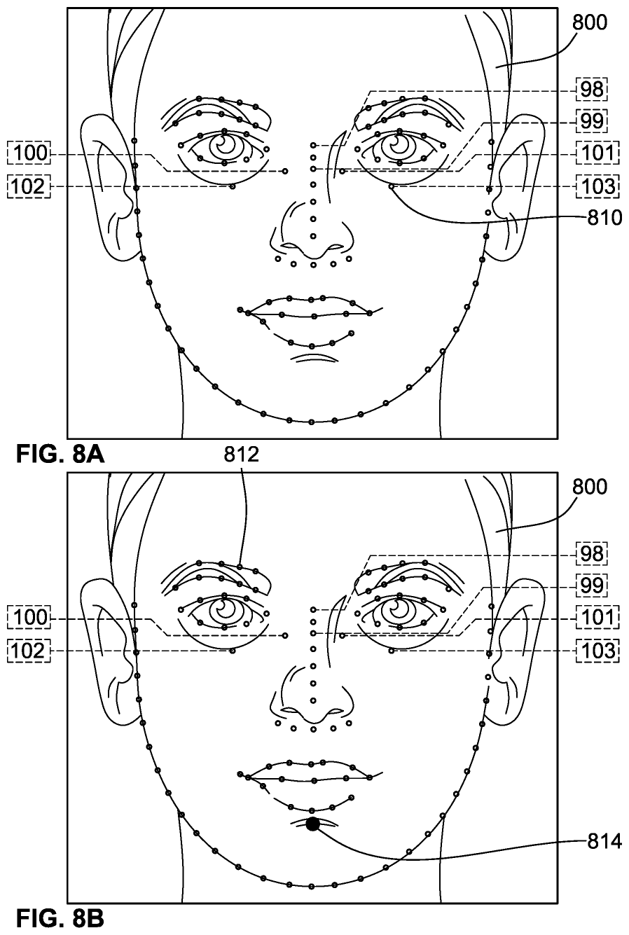

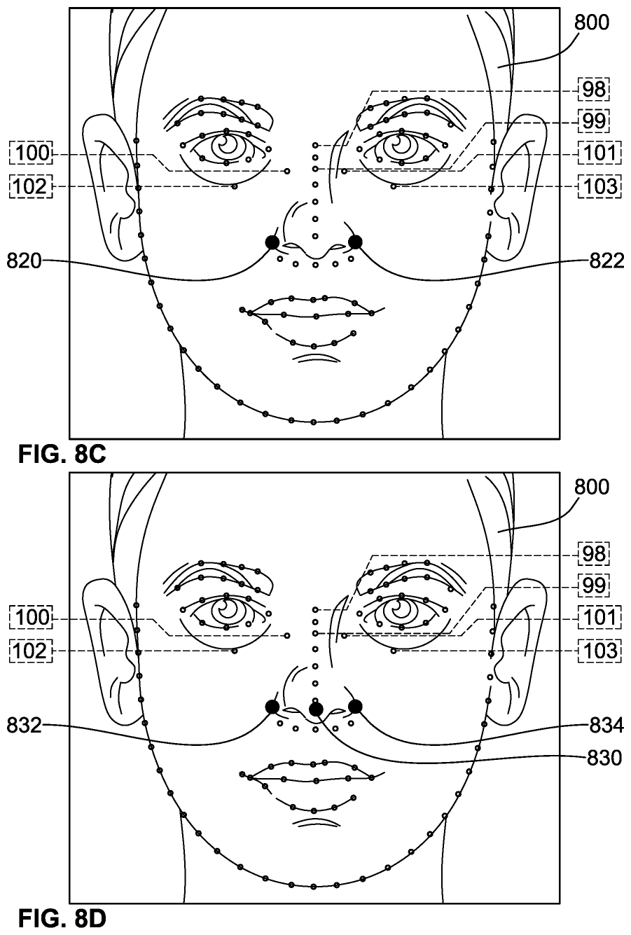

FIG. 8A is a facial image 800 such as one captured by the application described above that may be used for determining the face height dimension, the nose width dimension and the nose depth dimension. The image 800 includes a series of landmark points 810 that may be determined from the image 800 via any standardly known method. In this example, there are 100 Standard Cyborg landmark points that are identified and shown on the facial image 800. In this example, the method requires seven landmarks on the facial image to determine the face height, nose width and nose depth, for mask sizing relating to the users. As will be explained, two existing landmarks (based on Standard Cyborg landmarks for example) may be used. The location of the three dimensions requires five additional landmarks to be identified on the image via the processing method. Based on the imaging data and/or existing landmarks, new landmarks may be determined. The two existing landmarks that will be used include a point on the sellion (nasal bridge) and a point on the nose tip. The five new landmarks required include a point on the supramenton (top of the chin), left and right alar points, and left and right alar-facial groove points.

FIG. 8B shows the facial image 800 where the face height dimension (sellion to supramenton) is defined via landmark points 812 and 814. The landmark 812 is an existing landmark point on the sellion. The landmark point 814 is a point on the supramenton. The face height dimension is determined from the distance between the landmark points 812 and 814.

FIG. 8C shows the facial image 800 with new landmark points 820 and 822 to locate the nose width dimension. This requires two new landmarks, one on each side of the nose. These are called the right and left alar points and may correspond to the right and left alare. The distance between these points provides the nose width dimension. The alar points are different, but similar to, the alar-facial groove points.

FIG. 8D shows the facial image 800 with landmark points 830, 832 and 834 to determine the nose depth dimension. A suitable landmark is available for the landmark point 830 at the nose tip. The landmark points 832 and 834 are determined at the left and right sides of the nose. The landmark points 832 and 834 are at alar-facial grooves on the left and right sides of the nose. These are similar to alar points but at the back of the nose.

As explained above operational data of each RPT device may be collected for a large population of users. This may include usage data based on when each user operates the RPT device and the duration of the operation. Thus, compliance data such as how long and often a user uses the RPT device over a predetermined period of time, the therapy pressure used and/or whether the amount and manner of use of the RPT device is consistent with a user's prescription of respiratory therapy, may be determined from the collected operational data. For example, one compliance standard may be acceptable use of the RPT device by a user over a 90 day period of time. Leak data may be determined from the operational data such as analysis of flow rate data or pressure data. Mask switching data using analysis of acoustic signals may be derived to determine whether the user is switching masks. The RPT device may be operational to determine the mask type based on an internal or external audio sensor such as the audio sensor 4278 in FIG. 4B with cepstrum analysis as explained above. Alternatively, with older masks, operational data may be used to determine the type of mask through correlation of collected acoustic data to the acoustic signatures of known masks.

In this example, user input of other data may be collected via a user application executed on the computing device 230. The user application may be part of the user application 360 that instructs the user to obtain the facial landmark features or a separate application. This may also include subjective data obtained via a questionnaire with questions to gather data on comfort preferences, whether the user is a mouth or nose breather (for example, a question such as “Do you wake up with a dry mouth?”), and mask material preferences such as silicone, foam, textile, gel for example. For example, user input may be gathered through a user responding to subjective questions via the user application in relation to the comfort of the user interface. Other questions may relate to relevant user behavior such as sleep characteristics. For example, the subjective questions can include questions such as do you wake up with a dry mouth?, are you a mouth breather?, or what are your comfort preferences? Such sleep information may include sleep hours, how a user sleeps, and outside effects such as temperature, stress factors, etc. Subjective data may be as simple as a numerical rating as to comfort or more detailed response. Such subjective data may also be collected from a graphical user interface (GUI). For example, input data regarding leaks from a user interface that the user experienced during therapy may be collected by the user selecting parts of a user interface displayed on a graphic of the user interface on the GUI. The collected user input data may be assigned to the user database 260 in FIG. 7. The subjective input data from users may be used as an input for selection of the example mask type and size. Other subjective data may be collected related to the psychological safety of the user. For example, questions such as whether the user feels claustrophobic with that specific mask or how psychologically comfortable does the user feel wearing the mask next to their bed partner may be asked and inputs may be collected. If the answer to these questions is on the lower end indicating a negative response the system could recommend an interface from the interface database 270 that is less obtrusive such as a smaller mask than the user's existing mask, which may be a nasal cradle mask (a mask that seals to the user's face at an inferior periphery of the user's nose and leaves the user's mouth and nose bridge uncovered) or a nose-and-mouth mask that seals around the user's mouth and also at an inferior periphery of the user's nose but does not engage the nasal bridge (which may be known as an ultra-compact full face mask). Other questions around preferred sleeping position could also be factored in as well as questions around whether a user likes to move around a lot at night, and would the user prefer ‘freedom’ in the form of a tube up mask (e.g. a mask having conduit headgear) that may allow for more movement. Alternatively, if the user tends to lie still on their back or side a tube-down mask (e.g. a traditional style mask with the tube extending anteriorly and/or inferiorly from the mask proximate the user's nose or mouth) would be acceptable.

Other data sources may collect data outside of use of the RPT device that may be correlated to mask selection. This may include user demographic data such as age, gender or location; AHI severity indicating level of sleep apnea experienced by the user. Another example may be determining soft tissue thickness based on a computed tomography (CT) scan of a face. Other data may be the prescribed pressure settings for new users of the RPT device. If a user is prescribed a lower pressure such as 10 cm H2O, this may enable the user to wear a lighter mask suitable for lower pressures, resulting in greater comfort and/or less mask on the face as opposed to a full face with a very robust seal more suited for 20 cm H2O but may result in less comfort. However, if the user has a high pressure requirement e.g., 20 cm H2O the user may then be recommended a full face mask with a very robust seal.

After selection of the mask, the system continues to collect operational data from the RPT device 250. The collected data is added to the databases 260 and 270. The feedback from new and existing users may be used to refine recommendations for better mask options for subsequent users. For example, if operational data determines that a recommended mask has a high level of leaks, another mask type may be recommended to the user. Through a feedback loop, the selection algorithm may be refined to learn particular aspects of facial geometry that may be best suited to a particular mask. This correlation may be used to refine the recommendation of a mask to a new user with that facial geometry. The collected data and correlated mask type data may thus provide additional updating to the selection criteria for masks. Thus, the system may provide additional insights for improving selection of a mask for a user.

In addition to mask selection, the system may allow analysis of mask selection in relation to respiratory therapy effectiveness and user compliance. The additional data allows optimization of the respiratory therapy based on data through a feedback loop.

Machine learning may be applied to optimize both the mask selection process and provide correlations between mask types and increasing user compliance with respiratory therapy. Such machine learning may be executed by the server 210. The mask selection algorithm may be trained with a training data set based on the outputs of favorable operational results and inputs including user demographics, mask sizes and types, and subjective data collected from users. Machine learning may be used to discover correlation between desired mask sizes and predictive inputs such as facial dimensions, user demographics, operational data from the RPT devices, and environmental conditions. Machine learning may employ techniques such as neural networks, clustering or traditional regression techniques. Test data may be used to test different types of machine learning algorithms and determine which one has the best accuracy in relation to predicting correlations.

The model for selection of an optimal interface may be continuously updated by new input data from the system in FIG. 7. Thus, the model may become more accurate with greater use by the analytics platform.

As explained above, one part of the system in FIG. 6 relates to recommending an interface for users using the RPT devices. A second function of the system is a validation process that includes optimization of the interface selections. Once the user has been provided a recommended mask and has used it for a period of time, such as two days, two weeks, or another period of time, the system can monitor RPT device usage and collect other data. Based on this collected data, if the mask is not performing to a high standard as determined from adverse data indicating leaks, poor or dropping compliance, or unsatisfactory feedback, the system can re-evaluate the mask selection, and update the database 260 and machine learning algorithm with the results for the user. The system may then recommend a new mask to suit the new collected data. For example, if a relatively high leak rate is determined from data based off acoustic signatures or other sensors, the user may be jaw dropped during REM sleep, which may signal the need for a different type of interface such as a full face mask rather than an initially selected nasal only or smaller full face mask.

The system may also adjust the recommendation in response to satisfactory follow up data. For example, if operational data indicates an absence of leaks from a selected full face mask, the routine may recommend trying a smaller mask for a better experience. Tradeoffs between style, materials, variations, correlations with user preference to maximize user compliance with therapy may be used to provide follow up recommendations. The tradeoffs for an individual user may be determined through a tree of inputs that are displayed to the user by the application. For example, if a user indicates skin irritation is a problem from a menu of potential problems, a graphic with locations of the potential irritation on a facial image may be displayed to collect data such as the specific location of irritation from the user. The specific data may provide for better correlation to the optimal mask for the particular user.

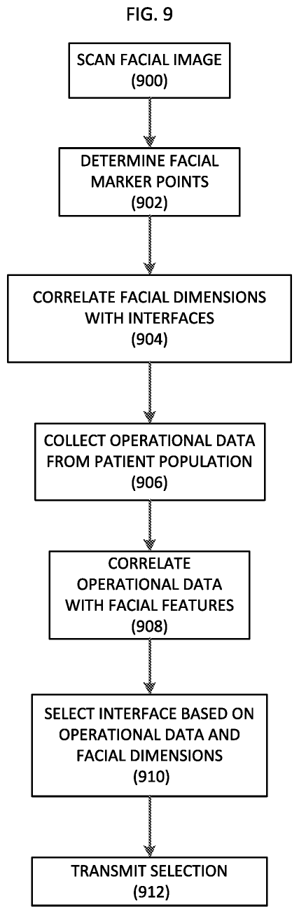

FIG. 9 is a flow diagram of the process of user interface selection that may be executed by an interface selection engine that may be executed by the server 210 in FIG. 7. The process collects an image of the face of the user (a facial image), which may then be stored on a storage device. The system 200 may comprise a facial profile engine operable to determine facial features of the patient based on the facial image. In some examples, any steps involving facial analysis may be performed by the facial profile engine. In this example, the face of the user is scanned via a depth camera on a mobile device such as the smart phone 234 or the tablet 236 in FIG. 7 to produce a 3D image of the face (900). Alternatively, 3D facial scan data may be obtained from a storage device with already scanned 2D or 3D facial images of the user. Landmark points are determined in a facial mesh from the facial 3D scan, and the key dimensions and collection of points in relation to user interface fit such as the face height, nose depth and nose width, are measured from the image (902). The measured key dimensions are then correlated with potential interfaces by size and type (904). For example, correlation may include fitting or non-rigid registration with a 3DMM. The irregular triangular surface mesh from the 3D scan is fitted with the 3DMM surface, which contains information about the face including locations and areas of key face regions.

Operational data is collected from a database in relation to respiratory devices and corresponding user interfaces (906). The operational data is analyzed in relation to desired outcomes such as reduced leaks and best compliance with respiratory therapy involving use of the respiratory therapy devices. The operational data for the desired outcomes for the determined facial dimensions and points is then correlated with the different sizes and types of interfaces available (908). A model is employed to select an appropriate interface based on the correlations between the operational data and the facial dimensions and points for desired results accounting for minimizing leaks, best fit, and best compliance with use of the respiratory therapy devices (910). Other factors may also be considered including user inputs such as satisfaction with the selected interface. The selected interface is then stored and sent to the user (912). As explained above, once the user uses the RPT device with the selected interface, the scan facial dimension data as well as data collected from the RPT device is added to the databases 260 and 270 to further refine the analysis of the selection process.

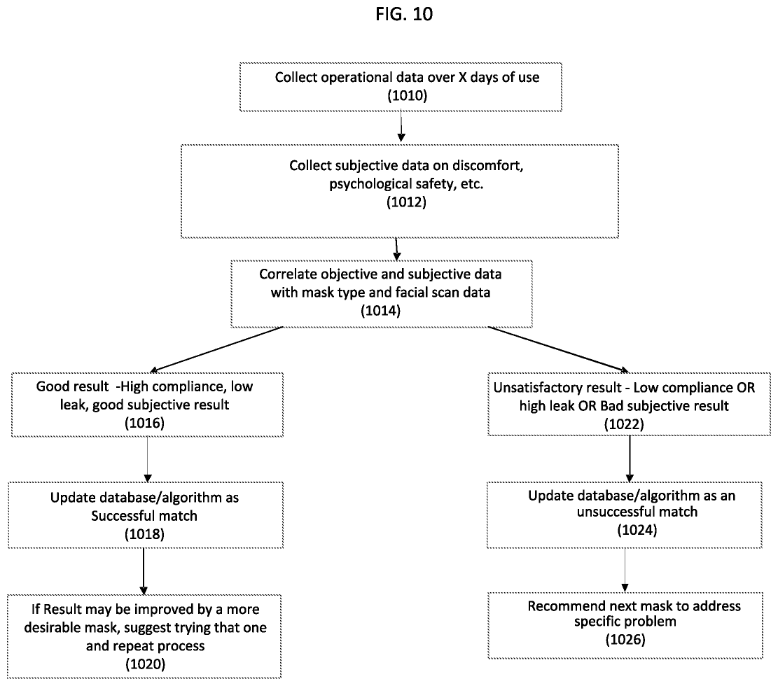

FIG. 10 is a follow up routine that may be run a specific time period or time periods after the initial selection of the interface detailed in FIG. 9. For example, the follow up routine may be run over the first two days of the use of the selected interface with the RPT device. As will be explained below, the routine in FIG. 10 may provide a recommendation for staying with the initially selected interface or switching to another type of interface. The collection of the additional objective and subjective data is recorded in the interface database 270. The routine in FIG. 10 thus records ongoing usage, switching and feedback data, and marks in mask types initially selected as “success” or “failure” in the interface database 270. This data continuously updates the example machine learning driven recommendation engine executed by the server 210.

The routine first collects operational data over a set period such as two days of use. (1010). For example, the system in FIG. 2 may collect complied objective data from two days of use such as time of use or leak data from the RPT device 250. Of course other appropriate periods greater than or less than two days may be used as a time period to collect operational data from the RPT device and other relevant data.

In addition, subjective feedback data such as seal quality/performance, comfort, general likes/dislikes etc., may be collected from an interface of the user application executed by the computing device 230 (1012). The subjective data may be collected via subjective questions asked via two-way communication devices or the client application executed on the computing device 230. The subjective data may thus include answers relating to discomfort or leaks and psychological safety questions, such as whether the user is psychologically comfortable with the mask. The subjective data may also include information provided by patients regarding tiredness experienced during waking hours.

The routine then correlates objective data and subjective data along with the selected mask size/type and the facial scan data of the user (1014). In the case of a good result, the routine will determine that the operational data shows high compliance, low leak, and a good subjective result data from the user (1016). The routine then updates the database and learning algorithm as a successful match with the correlated data (1018). The routine also analyzes the correlated data and determines whether the results may be improved by a more desirable mask (1020). The routine then suggests trying a more appropriate mask as per the routine in FIG. 9.

In the case of an undesirable result from the correlation (1014), the routine determines that the undesirable result is from at least one of low compliance, a high leak, or an unsatisfactory subjective result data (1022). The routine then updates the database 270 and learning algorithm as an unsuccessful match between the user data and the selected mask (1024). The routine then suggests trying a more appropriate mask as per the routine in FIG. 9 (1026).

User compliance with therapy may be determined to be high if the user is using the user interface as prescribed and based on a number of factors, which may be weighted equally or differently. Use of the user interface as often as the user has been prescribed to use it may count towards a high compliance with therapy. Using the user interface for sufficiently long periods, such as all night, rather than abandoning therapy during the night, may also count towards good compliance. Consistent adherence to their prescription (e.g. minimal missed nights) also count towards good compliance. Furthermore, if the user is using the prescribed therapy pressure, rather than a lower pressure, this may contribute towards an assessment of good compliance.

In some examples the user may be required to replace their user interface or components thereof according to a predetermined replacement schedule. For example, the user may be required to replace a cushion module of their user interface according to a predetermined interval based on the prescribed pressure and usage intensity, since wear on the seal and vents from use and cleaning may adversely affect performance and therefore effectiveness of the therapy. Timely replacement of components may count towards an assessment of good compliance with therapy in some examples of the present technology. If prescription data is not available then user compliance may be determined based on use of the user interface and the corresponding respiratory therapy device at least a predetermined number of nights each week (e.g. at least 4, 5, 6 or 7 nights), for at least a predetermined number of hours e.g. 4, 5, 6 or 7, with a consistently high therapy pressure (e.g. at least 4, 6, 8 or 10 cm H2O) and/or with timely replacement of the user interface or components thereof.

As described above, there are multiple ways in which a user may be compliant with therapy. In some examples of the present technology, the degree of compliance is determined to be either “not compliant” or “compliant” based on whether or not one or more criteria are satisfied. In some examples the compliance may be one of “low”, “medium” or “high”. In further examples compliance may represented as a score out of 100%. A good level of compliance may not necessarily be 100%, and may instead be at least 75% or 80%, in some examples.

The above described system and mobile device may also be used for determining proper fit once the physical user interface such as a mask is made available to the user.

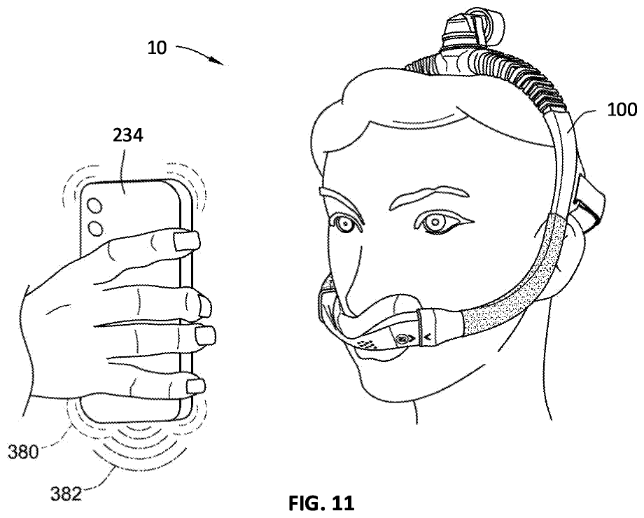

FIG. 11 is a perspective view of a user such as the user 10 controlling a user device such as the smart phone 234 in FIG. 7 to collect sensor data associated with a current fit of a user interface such as the mask 100, according to some implementations of the present disclosure. For example, user 10 can be a new user of a respiratory therapy system such as that in FIG. 1. User 10 can have just recently donned the user interface 100, and is now directing one or more sensors of the user device 234 towards the user's face. The mask 100 may be one that is selected based on the above described operational data and facial data. While depicted as a smart phone 234 in FIG. 11, any suitable user device can be used.

To acquire measurements, the user 10 can press the appropriate button or otherwise interact with the smart phone 234 to start a measurement process. Once started, the smart phone 234 can optionally provide stimuli (e.g., prompts and/or instructions) to the user 10 indicating different actions the user 10 should take or stop in order to effect the desired measurements. For example, the prompts and/or instructions can include text instructions such as “hold the phone at face-height and slowly move in a figure eight pattern”; auditory prompts 382, such as a particular chime playing when the measurement process is complete; and/or haptic feedback 380, such as an increasing vibration pattern signaling the user 10 to slow down movement of the smart phone 234. The use of non-visual prompts (e.g., auditory prompts, haptic feedback, and the like) can be especially useful when the smart phone 234 must be held in an orientation that prohibits the user 10 from viewing the display of the smart phone 234. In some cases, prompts and/or instructions can be presented as overlays on an image of the user 10, such as in the form of an augmented reality overlay. For example, icons, highlighting, text, and other indicia can be overlaid on an image (e.g., live or non-live) of the user 10 to provide instructions for how to perform a measurement process.

The smart phone 234 can include one or more sensors. In some cases, the user 10 can be instructed to hold the smart phone 234 in a particular orientation to ensure the desired one or more sensors are acquiring the desired data. For example, when an infrared sensor on a front of a smart phone is being used (e.g., an infrared sensor used to unlock the phone), the user 10 may be instructed to hold the phone with its face facing the smart phone 234, such that the infrared sensor faces the user's face and acquires data of the user's face. In another example, the smart phone 234 may include a LiDAR sensor on its rear side, in which case the user 10 may be instructed to hold the phone with its face facing away from the user 10, such that the LiDAR sensor faces the user's face and acquires data of the user's face. In some cases, the user 10 can be instructed to take multiple measurements with the smart phone 234 positioned in different orientations.

The smart phone 234 can provide feedback regarding the current fit of the user interface 100, whether during a measurement process (e.g., real-time feedback) and/or after conclusion of a measurement process (e.g., non-real-time feedback). In an example, while the user 10 is holding the smart phone 234 to acquire measurements, the smart phone 234 may provide feedback indicating that the user 10 should make an adjustment to improve the current fit of the user interface 100, such as tightening a particular strap. In such an example, the user 10 may make the adjustment while continuing to hold the smart phone 234 such that it can continue to acquire measurements of the user's face and/or user interface 100. In such cases, the smart phone 234 may provide dynamic feedback showing how the current fit is improving or otherwise changing.

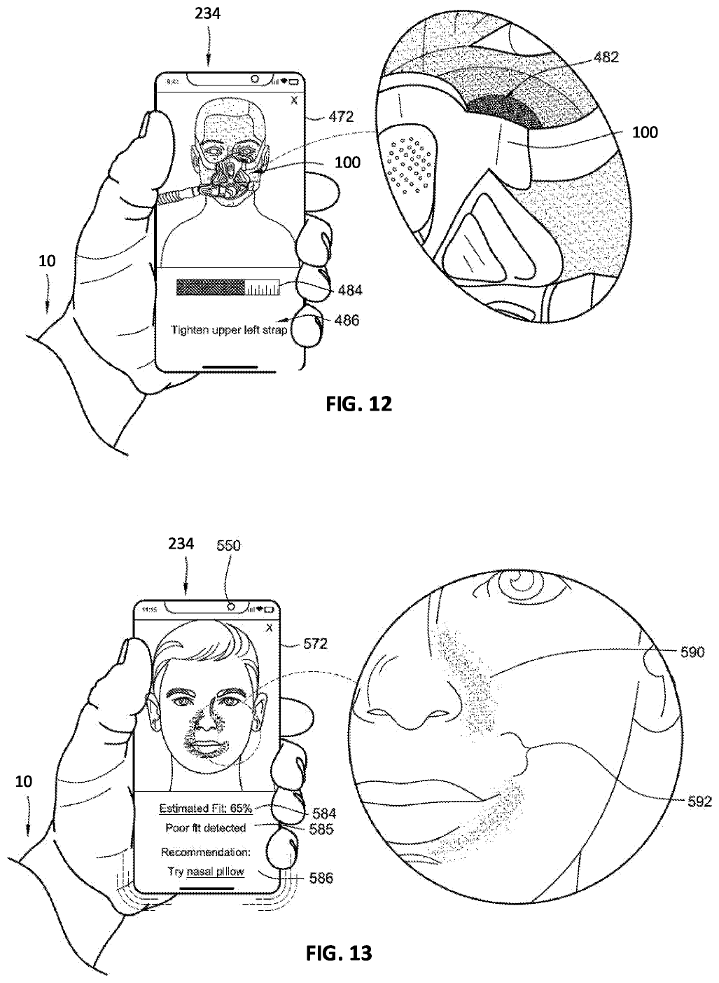

FIG. 12 is a user's view of the smart phone 234 being used to identify a thermal characteristic associated with a current fit of the user interface 100, according to some implementations of the present disclosure. The smart phone 234 and user interface 100 can be any suitable user device and user interface. The view depicted in FIG. 12 can be made during a measurement process (e.g., viewed live, such as in real-time) or can be after measurements have been taken (e.g., viewed after a measurement process is complete).

The user 10 can be holding a user device (e.g., smart phone 234) such that they are able to see a display device 472 (e.g., display screen) of the smart phone 234. The display device 472 can depict a graphical user interface (GUI) that can provide feedback associated with the current fit of the user interface 100. The GUI can include an image of the user 10 wearing the user interface 100. The image can be acquired by an infrared sensor and can be a thermal map of the user 10 wearing the user interface 100. The thermal map can depict localized temperatures at different points on the user's face and user interface 100.

As shown in the enlarged view, a region 482 of the user's face near the seal between the user interface 100 and the user's face is shown as being substantially colder than surrounding regions of the user's face. This colder region 482 can be indicative of an unintentional air leak, as air leaking from the seal of the user interface 100 can cool down the user's skin by an amount perceptible by the IR sensor.

In some cases, the GUI can provide feedback in the form of a score 484 associated with the current fit of the user interface 100. For example, the score 484 can be depicted as a filled bar that can be filled between 0% and 100%. As depicted in FIG. 12, the score 484 of the current fit of the user interface 100 is currently approximately 65%.

In some cases, the GUI can provide feedback in the form of textual instructions 486 to improve the current fit. The textual instructions 486 can provide instructions for the user 10 to take an action to improve the current fit, such as tightening the upper left strap as depicted in FIG. 12. The instruction to tighten the upper left strap was selected because doing so should reduce or eliminate the air leak detected by the thermal difference at region 482.

FIG. 13 is a user's view of a user device such as the smart phone 234 being used to identify a contour-based characteristic associated with a current fit of a user interface, according to some implementations of the present disclosure. The user device can be any suitable user device, such as those shown in FIG. 7.

A display device 572 (e.g., display screen) of the smart phone 234 can display a GUI that includes a live image of the user 10. To create the live image of the user 10, a camera 550 of the smart phone 234 can be pointed towards user 10.

In the view depicted in FIG. 13, the user 10 has just recently removed a user interface, leaving an indentation 590 surrounding portions of the user's face. This indentation 590 can be detected through visual sensing (e.g., sensing a color of the indentation 590 or a color difference between the indentation 590 and adjacent surface of the user's face), ranging sensing (e.g., sending a localized difference in depth between the indentation 590 and adjacent surface of the user's face), and/or the like. As depicted, a region 592 of the indentation 590 is less pronounced than other regions. This region 592 can be identified as a region where the user interface is not sufficiently pressed against the skin of the face, thus not establishing an effective seal.

In some cases, the GUI can provide feedback in the form of a score 584 associated with the current fit of the user interface. For example, the extent of the region 592 can indicate that the current fit of the user interface (e.g., the fit of the user interface that was removed prior to collecting the sensor data that led to identification of region 592) is not optimal. A score 584 can be generated and depicted, such as via a numerical score of “65%.” In some cases, an alert can additionally be provided, such as an alert 585 indicating that poor fit is detected. In some cases, the GUI can provide feedback in the form of textual instructions 586 to improve the current fit. The textual instructions 586 can provide instructions for the user 10 to take an action to improve the current fit, such as by using a different type of user interface (e.g., a nasal pillow's mask instead of a full face user interface). The instruction to use a nasal pillow's mask instead of a full face user interface may have been selected because of the nature of the detected poor fit and/or one or more previous attempts to improve fit of the current user interface. For example, if a user attempts to improve the fit of the current user interface more than a threshold number of times, the system may determine that it may be prudent to try using a different type of user interface to establish a good fit. The available user interfaces may be selected using the routine that incorporates both facial data and operational data from RPT devices described above.

While indentation 590 is being detected in FIG. 13 as a detected contour on the user's face, in some cases a color change in the user's face (e.g., blanching, or color change as some blood is urged out of tissue near the surface of the skin where the seal of the user interface rests on the surface of the skin) can be detected to identify where and how the seal of the user interface engages the user's face.

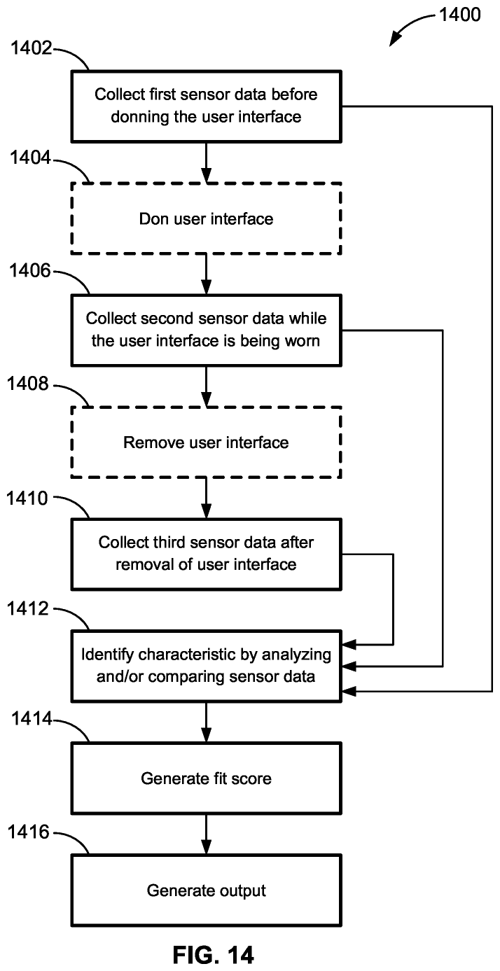

FIG. 14 is a flowchart depicting a process 1400 for evaluating fit of a user interface across user interface transition events, according to some implementations of the present disclosure. The user interface can be any suitable user interface, such as user interface 100 of FIG. 1. The process 1400 can be performed using a control system such as the processor for the smart phone 234 or the processors of a server such as the server 210 in FIG. 7. Sensor data collected during process 1400 can be collected from one or more sensors (e.g., one or more sensors of the RPT device 40 in FIGS. 4A-4C), of which one, more than one, or all can be incorporated in and/or coupled to a user device (e.g., smart phone 234 of FIG. 7). Examples of user interface transition events include donning the user interface, removing the user interface, adjusting the user interface (e.g., making an adjustment to an adjustable part of the user interface, such as an adjustable strap; moving the user interface to a different position or orientation on the user's face; or the like), and adjusting a respiratory therapy system such as the RPT device 40 coupled to the user interface (e.g., turning on or turning of the respiratory therapy system; adjusting parameters, such as humidity or heating, of the respiratory therapy system; or the like).

At block 1402, first sensor data can be collected before donning the user interface. The first sensor data can be collected of the user's face, and optionally the user interface (before being donned by the user).

In some cases, first sensor data can be used at block 1412 to identify one or more characteristics associated with a potential fit of a user interface. For example, based on characteristics identified from solely first sensor data, the system may determine that the user would be best suited to use a nasal pillow's mask rather than a full face user interface (e.g., if particularly substantial contours are detected around where a full face user interface seal would normally sit or if a particularly thick beard is detected around where a full face user interface seal would normally sit).

In some cases, however, first sensor data is used at block 1412 to compare with other sensor data. For example, in some cases, the first sensor data can establish a baseline for future comparison, such as a baseline contour map of the face, a baseline thermal map of the face, a baseline detection of one or more features of the face (e.g., detection of eyes, mouth, nose, ears, and the like), and the like. First sensor data can be considered to be sensor data that is associated with a current fit of a user interface, such as if collected prior to donning the user interface for the purposes of establishing a baseline against which further sensor data is compared to assess the fit of the user interface when worn. For example, such data may be stored from the facial scan performed as explained above in reference to FIGS. 8A-8D.

At block 1404, the user interface can be donned. Donning the user interface can involve the user placing the user interface on the user's face as if they were using it (e.g., for respiratory therapy). In some cases, donning the user interface at block 1404 can include making an adjustment to the user interface prior to donning the user interface, although that need not always be the case. In such cases where an adjustment to the user interface is made prior to donning the user interface, further analysis of the sensor data can be compared to historical sensor data, historical characteristic data, historical fit scores, and the like. For example, donning the user interface at block 1404 can occur immediately following a prior evaluation of user interface fit that identified an action to be taken to improve fit. In such an example, the user can take that action as part of block 1404, then compare the resultant fit of the user interface with the prior fit of the user interface to determine whether or not the fit has improved.

In some cases, donning the user interface at block 1404 can include wearing the user interface for a threshold duration of time. For example, if an evaluation of user interface fit is being made by comparing sensor data from before and after wearing a user interface, process 1400 may proceed from block 1404 to block 1408. In such cases, to ensure the user interface is worn for a sufficient duration of time to affect the user's face (e.g., sufficient duration of time to establish indentations or color changes in the user's face), donning the user interface at block 1404 can include wearing the user interface for the threshold amount of time (e.g., at least 10 seconds, 20 seconds, 30 seconds, 40 seconds, 50 seconds, 1 minutes, 1.5 minutes, 2 minutes, 5 minutes, 10 minutes, or 15 minutes).

At block 1406, second sensor data can be collected while the user interface is being worn (e.g., worn on the user's face). The second sensor data can be collected of the user's face and the user interface as worn by the user. In some cases, second sensor data can be collected while one or more adjustments are being made to the user interface and/or to a respiratory therapy system coupled to the user interface. In such cases, the second sensor data taken over time can be used to detect changes in identified characteristics at block 1412, which can be used in the evaluation of the current fit of the user interface. In an example, thermal data collected at block 1406 can identify a change in temperature of a region of the user's face outside of and adjacent to the user interface as a heater of the respiratory therapy system in FIG. 1 is engaged, indicating that an unintentional air leak may exist at the seal of the user interface near that region of the user's face. In such an example, additional sensor data, such as audio data collected by the audio sensor 4278 in FIG. 4C, can be used to confirm the existence of the leak (e.g., via detecting a characteristic acoustic signal associated with an unintentional leak, such as an audible or inaudible signal).

At block 1408, the user interface can be removed. Removal of the user interface can be performed while the one or more sensors are still collecting sensor data, although that need not always be the case.

At block 1410, third sensor data can be collected after the user has removed the user interface. The third sensor data can be similar to first sensor data from block 1402, except for where the sensor data is affected by changes in the user's face due to having worn the user interface.

Collecting sensor data at block 1402, block 1406, and block 1410 can include collecting the same types of sensor data—including sensor data from the same or different sensors—and/or different types of sensor data. For example, first and second sensor data collected at blocks 1402 and 1410, respectively, may each include ranging sensor data and image data in the visual and IR spectrums, whereas second sensor data collected at block 1406 may include audio data and image data in the visual and IR spectrums.

At block 1412, one or more characteristics are identified from the sensor data (e.g., first, second, and/or third sensor data). Identifying a characteristic at block 1412 can include analyzing a given set of sensor data (e.g., second sensor data analyzed alone) and/or comparing sets of sensor data (e.g., first sensor data as compared with third sensor data). Identifying a characteristic at block 1412 can include identifying characteristics that are indicative of quality of fit of the user interface. For example, some characteristics may indicate a poor fit of the user interface, whereas other characteristics may indicate a good fit of the user interface. In an example, a characteristic that is the sound indicative of an unintentional air leak may be indicative of a poor fit, whereas a characteristic that is a thermal map of the user's face wearing the user interface showing consistent and/or expected temperatures on the surface of the user's face adjacent the user interface may be indicative of a good fit.

A characteristic can be output in a suitable fashion, including as a value (e.g., a numerical value or Boolean), a set of values, and/or a signal (e.g., a set of values over time). In an example, a characteristic associated with the thermal mapping of a user's face while wearing a user interface can be output as i) a Boolean value of whether or not a localized change in temperature above a threshold is detected (e.g., change in temperature over time or across a distance on the surface of the face); ii) a set of temperature values taken at different locations on the user's face around the circumference of the seal of the user interface; iii) a thermal image or video of the user's face; iv) or any combination of i-iii, as well as others.

In some cases, at block 1414, a score associated with the fit of the user interface can be generated. Generating the fit score can include analyzing the characteristics identified at block 1412 and generating a score from the characteristics. In some cases, the score can be based on or based at least in part on a calculation using the one or more characteristics of block 1412 as inputs, and/or sensor data from blocks 1402, 1406, and/or 1410.

In some cases, the score can be calculated using a machine-learning algorithm using the one or more characteristics of block 1412 and/or sensor data from blocks 1402, 1406, and/or 1410 as input. Such a machine-learning algorithm can be trained using characteristics and/or sensor data associated with a training set of fit evaluations of users wearing a user interface. The fit evaluations in the training set can be based on subjective evaluation of the users, objective values collected with the use of other equipment (e.g., laboratory sensors and equipment, such as user interfaces outfit with specialized sensors and/or specialized sensing equipment), and the like.

At block 1416, output can be generated. The output, or output feedback, can include any suitable output for relaying information regarding the current fit of the user interface. The output can be based on the one or more characteristics of block 1412; sensor data from blocks 1402, 1406, and/or 1410; and/or the fit score from block 1414. In some cases, the output can be the fit score generated at block 1414. In some cases, the output can be raw or processed sensor data from blocks 1402, 1406, and/or 1410 (e.g., a thermal image presented to the user or a portion of an audio recording).

In some cases, the output can be an instruction or suggestion for an action that is selected to improve the current fit of the user interface. For example, if a characteristic identified at block 1412 is indicative that an unintentional air leak exists at a certain location with respect to the user's interface, the output can include an instruction to make an adjustment to the user interface (e.g., tighten a strap) to reduce the unintentional air leak, thus improving the fit of the user interface. In some cases, improvement to the fit of a user interface can be based on the current fit score generated at block 1414 and a past or future fit score generated at a past or future instance of block 1414.

In some cases, the output selected to improve the current fit can be selected using a machine-learning algorithm using as input the one or more characteristics of block 1412; sensor data from blocks 1402, 1406, and/or 1410; and/or the fit score from block 1414. Such a machine-learning algorithm can be trained using characteristics, sensor data, and/or fit scores associated with a training set of fit evaluations of users wearing a specific user interface. The training data can include information about adjustments made, such as adjustments made to the user interface, adjustments made to the user's face (e.g., shaving a beard), and/or selection of different user interfaces.

In an example in which an evaluation of user interface fit is made by comparing sensor data from before a user interface is worn with sensor data while the user interface is worn, process 1400 include only blocks 1402, 1404, 1406, 1412, and 1414. In another example, in which an evaluation of current fit is made while the user is wearing the user interface, process 1400 includes only blocks 1406, 1412, and 1414. In another example, in which an evaluation of current fit of the user interface is made by comparing characteristics of the user's face before and after wearing a user interface, process 1400 includes only blocks 1402, 1404, 1408, 1410, 1412, and 1414. Other arrangements can be used. Additionally, in some cases, aspects presented as separate blocks can be incorporated into one or more other blocks. For example, in some cases, generating a fit score at block 1414 occurs as part of generating output at block 1416. In another example, in some cases, process 1400 can proceed from block 1412 to block 1416 without generating a fit score at block 1414.

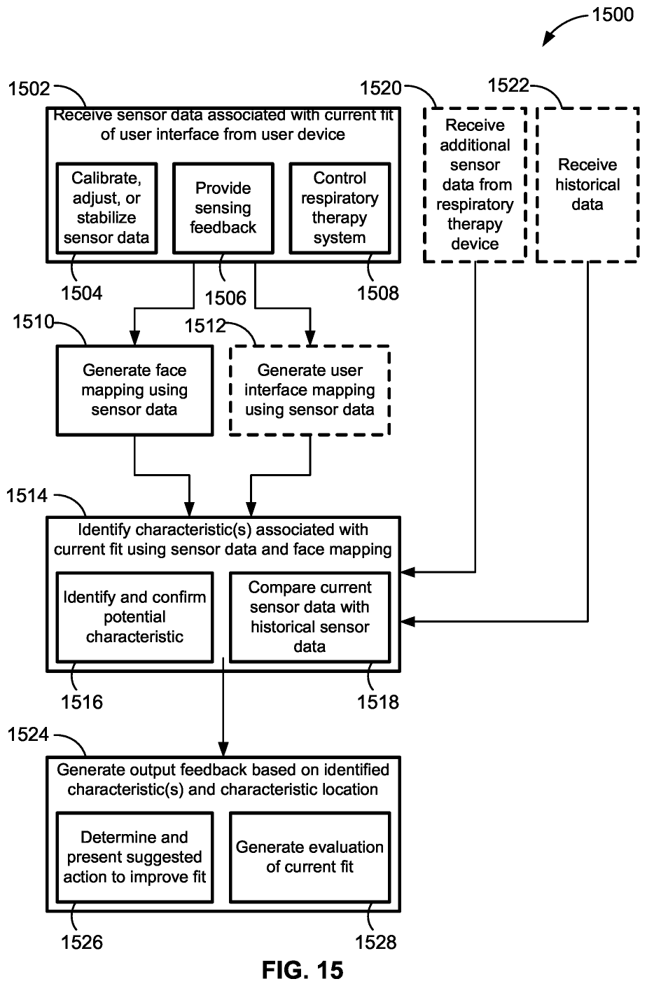

FIG. 15 is a flowchart depicting a process 1500 for evaluating fit of a user interface, according to some implementations of the present disclosure. The user interface can be any suitable user interface, such as user interface 100 in FIG. 1. The process 1500 can be performed using the control system, such as the processor for the smart phone 234 or the processors of a server such as the server 210 in FIG. 7.

At block 1402, sensor data associated with a current fit of the user interface is received, such as from the user device. The sensor data received at block 702 can have been collected from one or more sensors (e.g., one or more sensors/transducers 4270 of the RPT device 40 in FIGS. 4A-4C), of which one, more than one, or all can be incorporated in and/or coupled to a user device (e.g., smart phone 234 of FIG. 7). The sensor data can be collected at the same or different frequencies, with any suitable resolution. In some cases, the frequency of detection can be based on the sensor used. For example, image data in the visual spectrum may be acquired at a framerate of 25-60 frames per second (fps) or more, whereas thermal data at IR frequencies may be acquired at a framerate of 10 fps or less.

In some cases, receiving the sensor data at block 1502 can include calibrating, adjusting, or stabilizing sensor data at block 1504. Calibrating, adjusting, or stabilizing sensor data can include making adjustments to sensor data to account for undesired artifacts in the data. Calibrating, adjusting, or stabilizing sensor data can include making adjustments using a portion of the sensor data. In an example, image data can be stabilized at block 1502, such as by applying image stabilizing software to the image data or by applying inertia data acquired from an inertia measurement unit (IMU) or sub-sensor of the smart phone. In some cases, stabilizing sensor data can include receiving image stabilization information associated with a first portion of sensor data (e.g., image stabilization information associated with image data from a visual spectrum camera) and applying that stabilization information to a second portion of sensor data (e.g., sensor data from another sensor, such as thermal data from an IR sensor). In an example, image stabilization information associated with stabilization of visual spectrum camera data (e.g., as obtained via image stabilizing software and/or inertia data) can be applied to thermal data from an IR sensor, thus allowing the thermal data to be stabilized beyond what may otherwise be possible when using the thermal data alone.

Calibration can occur by acquiring sensor data of a known or nominal object or event. For example, calibration of exposure and/or color temperature in image data can be achieved by first collecting reference image data from a calibration surface (e.g., a surface of a known color, such as a white balance card), then using that reference image data to adjust settings of the sensor acquiring the image data and/or adjusting the image data itself.

Adjustment can occur by using a portion of the sensor data to inform adjustments made to other sensor data. For example, a thermal imager (e.g., IR sensor) can collect thermal data to generate a thermal map of the user's face. The thermal map may identify localized temperatures at various locations on the user's face, as well as some surfaces in the surrounding environment, such as a surface behind the user. In such an example, the thermal data may indicate that the surface behind the user is 19° C. However, ambient temperature data collected from a separate temperature sensor may indicate that the surface behind the user and/or the ambient room temperature is closer to 21° C. Therefore, the system may automatically adjust the thermal data acquired from the thermal imager such that the temperature sensed for the surface behind the user is equal to that measured by the separate temperature sensor (e.g., 21° C.). Such an adjustment may therefore also carry over to localized temperature values at the various locations on the user's face.

In another example, sensor data from one or more sensors can be used to correlate with and coordinate sensor data from one or more other sensors. For example, image data from a visual spectrum camera can be correlated with thermal mapping data from an IR sensor. In such an example, certain detected features in the image data (e.g., eyes, ears, nose, mouth, user interface vent, etc.) can be compared with similar detected features in the thermal mapping data. Thus, if the image data and thermal mapping data are not collected with the same scale and field of view, the data can nevertheless be correlated together. For example, all pixels of the thermal mapping data can be adjusted (e.g., stretched in X and/or Y directions) so that the locations of features detected in the image data match the locations of correlated features in the thermal mapping data.

In some cases, adjusting sensor data can include adjusting sensor data with portions of the sensor data related to i) movements of the user device, ii) inherent noise of the user device, iii) breathing noise of the user, iv) speaking (or other vocalization) noise of the user, v) changes in ambient lighting, vi) detected transient shadows cast on the face of the user, vii) detected transient colored light cast on the face of the user, or viii) any combination of i to vii.

Other techniques can be used to calibrate, adjust, or stabilize, or otherwise modify the sensor data based on a portion of the sensor data and/or other data (e.g., historical sensor data, sensor data acquired form a sensor not coupled to the system, and the like).

In some cases, at block 1506, sensing feedback can be provided as part of receiving sensor data at block 1502. Providing sensing feedback can include presenting feedback to the user regarding the process of receiving sensor data at block 1502. In a first example, sensing feedback can include text or images on a display that indicate instructions regarding the ongoing collection of sensor data. For example, the sensing feedback may take the form of an outline or other indicia designating a region of the screen overlaid on a live camera feed of a user-facing camera, in which case the user is instructed to keep their face within the outline or region of the screen.

In another example, receiving sensor data at block 1502 can occur for a set duration of time or until certain sensor data has been successfully obtained. In such a case, the user may need to hold the user device in a particular orientation. Once duration of time has elapsed or the certain sensor data has been obtained, the user device can present sensing feedback 1506 to indicate that the user may stop holding the user device in the particular orientation (e.g., with the display of the user device facing away from the user). Sensing feedback can be in the form of visual and/or non-visual cues (e.g., stimuli, such as prompts, instructions, notifications, etc.). In some cases, the use of non-visual cues (e.g., audio cues or haptic feedback) can be especially useful, such as if the display of the user device is not currently visible to the user.

In some cases, providing sensing feedback at block 1506 can include presenting instructions to the user to perform certain actions to evoke an effect detectable by the one or more sensors. For example, the user may be instructed to hold their breath for a certain duration of time, to smile at the camera, to chew, to yawn, to speak, to put on and/or remove the user interface, or the like. The detectable effect can be used in the detection of characteristics at block 1514. In an example, the detectable effect can be an amount of movement of the user interface with respect to the user's face as the user speaks, chews, or yawns. In such an example, excessive movement of the user interface may indicate a poor fit. In some cases, receiving sensor data at block 1502 can include receiving a completion signal associated with the user's completion of the action instructed at block 1506. Examples of completion signal detection include sensing a button press (e.g., the user pressing a “completed” button) and automatically detecting completion of the action (e.g., via camera data).

In some cases, providing sensing feedback at block 1506 can include providing instructions for the user to move to a desired location (e.g., move indoors or move to a well-lit room), adjust the environment (e.g., turn on or off lights in the room), and/or adjust their orientation or pose (e.g., sit upright).

In some cases, receiving sensor data at block 1502 can include controlling a respiratory system at block 1508. Controlling the respiratory therapy system at block 1508 can include transmitting a control signal to a respiratory therapy device coupled to the user interface. The control signal, when received by the respiratory therapy device, can cause the respiratory therapy device to adjust a parameter or take some action. For example, a control signal can cause the respiratory therapy device to turn on and/or off; to supply air at a given pressure or a preset pattern of pressures; to activate and/or deactivate a heater and/or humidifier; and/or take any other suitable action.

At block 1510, a face mapping can be generated using the sensor data. The face mapping can be generated using any suitable sensor data, such as ranging data (e.g., from a LiDAR sensor or IR sensor), image data (e.g., from a camera), and/or thermal data (e.g., from an IR sensor). The face mapping can identify one or more features of the user's face, such as eyes, nose, mouth, ears, irises, and the like. Face mapping can include measuring distances between any combination of the identified feature(s). The resultant face map can be a two-dimensional or three-dimensional face mapping. Alternatively, the face mapping and resulting data may be taken from stored data collected from the facial scan performed as explained above in reference to FIGS. 8A-8C.

In some cases, the face mapping is a contour map, indicating contours and elevations associated with the user's face. In some cases, the face mapping is a thermal map, indicating localized temperatures at different locations on the user's face.

In some cases, generating the face mapping at block 1510 can include identifying a first individual and one or more additional individuals. In such cases, generating the face mapping can include selecting the first individual (e.g., based on the first individual being closest to the one or more sensors or user device, based on a comparison to a previously recorded image or characteristic of the first user, or the based on other such analysis) and proceeding with face mapping generation using one or more features detected on the first individual's face.

In some cases, at optional block 1512, a user interface mapping can be generated using the sensor data. The user interface mapping can be generated using any suitable sensor data, such as ranging data (e.g., from a LiDAR sensor or IR sensor), image data (e.g., from a camera), and/or thermal data (e.g., from an IR sensor). The user interface mapping can identify one or more features of the user interface, such as vents, contours, conduits, conduit connections, straps, and the like. User interface mapping can include measuring distances between any combination of the identified feature(s). The resultant user interface map can be a two-dimensional or three-dimensional user interface mapping.

In some cases, generating a face mapping at block 1510 can include generating a user interface mapping at block 1512.

At block 1514, one or more characteristics associated with the current fit are identified using the sensor data from block 1502 and face mapping from block 1510. In some cases, identification of a characteristic at block 1514 can include the use of the interface mapping generated at block 1512.

Any suitable characteristic can be identified. Characteristics can include characteristics of a user's face, characteristics of the interaction between the user's face and the user interface, characteristics of the user interface, and characteristics of the environment. In some cases, a characteristic can be an identifiable aspect of the user's face, of the user interface, of the interaction between the user's face and the user interface, or of the environment, which can be detected from the sensor data, and which has probative value for determining a quality of fit of the user interface.

In some cases, a characteristic can be associated with a location, although that need not always be the case. A characteristic that is associated with a location can be associated with a location on or with reference to the face mapping and/or the user interface mapping. For example, an example characteristic can be an audio signal indicative of an unintentional air leak (e.g., an audio signal having a characteristic frequency profile associated with an unintentional air leak) or the unintentional air leak itself. In some cases, that characteristic can be used on its own (e.g., the presence or absence of an unintentional air leak can be used, such as to generate a fit score), or in combination with location information associated with the characteristic. In such an example, the unintentional air leak can be detected to be at a certain location on the user's face (e.g., between the nose and the left cheek as seen in FIG. 12), in which case the location information can be indicative of a location on the face mapping or user interface mapping that correlates with where the unintentional air leak exists on the user's face. The addition of location information along with the user interface can facilitate providing useful information, such as a more accurate fit score and/or more accurate instructions to improve the fit.

In some cases, a set of available characteristics can be predetermined. In such cases, identifying a characteristic at block 1514 can include analyzing the sensor data and/or the face mapping to determine which, if any, of the set of available characteristics is detected. For example, a set of available characteristics can include a localized temperature rebound on the face of the user, a localized color rebound on the face of the user, and a localized temperature on the user interface. In such an example, if the sensor data does not include thermal data (e.g., if no IR sensor or temperature sensor is available), the only characteristic out of that list that might be determined is the localized color rebound, which may be detected from image data collected by a camera. In another example, if there is sufficient thermal data, the localized temperature rebound and localized temperature of the user interface may additionally be detected.

In some cases, characteristics can be beneficial characteristics or detrimental characteristics. Beneficial characteristics can be characteristics that are associated with a good fit of the user interface. For example, a characteristic that is a localized contour on the face of the user in the shape of a seal of a user interface can be indicative that the user interface is maintaining a good fit with the user's face. Thus, the presence of, or greater presence of, such a characteristic can be beneficial. By contrast, a detrimental characteristic can be associated with poor fit of the user interface. For example, a characteristic that is a localized temperature change over a region of the face of the user can be indicative that cold air is flowing over that region of the user's face, thus indicating an unintentional air leak, which is associated with a poor fit. Thus, the presence of, or greater presence of, such a characteristic can be detrimental.

One example characteristic is a localized temperature on the face of the user. This characteristic can be a detected temperature on the face of the user, which can be compared with previously detected temperatures at that same location (e.g., a previous time the user was wearing the user interface) or current temperatures detected at adjacent locations. For example, if a cold spot is detected near a seal of a user interface, but is surrounded by warmer temperatures, that cold spot may be indicative of an unintentional air leak.

Another example characteristic is a localized temperature rebound on the face of the user. A localized temperature rebound can include a change in temperature over a period of time at a certain location (e.g., a certain location on the face mapping). The localized temperature rebound may follow a transient event, such as a user interface transient event (e.g., removal of the user interface), a user transient event (e.g., moving from a warm room to a cooler room), a respiratory therapy system transient event (e.g., engaging the heater(s) of the respiratory therapy system such as the heater of the flow generator, the heater of the humidifier and/or the heater of the conduit), or another transient event. For example, detection of a longer temperature rebound time at certain locations with respect to the face mapping after removal of the user interface may indicate a poor fit. A “rebound” of a measured property (e.g. temperature, color, contour etc.) may comprise a change in the measured property following a transient event, such as donning, doffing or adjusting the user interface, starting or stopping a flow of pressurized air from the RPT device or another event which produces a change in the measured property over time, for example.

Another example characteristic is a localized color on the face of the user. This characteristic can be a detected color on the face of the user, which can be compared with previously detected colors at that same location (e.g., a previous time the user was wearing the user interface) or current colors detected at adjacent locations. For example, if a redder spot is detected near a seal of a user interface, but is surrounded by whiter colors, that redder spot may be indicative of an unintentional air leak (e.g., due to the seal not pressing against the user's face as much and/or due to irritation from flowing air).

Another example characteristic is a localized color rebound on the face of the user. A localized color rebound can include a change in color over a period of time at a certain location (e.g., a certain location on the face mapping). The localized color rebound may follow a transient event, such as a user interface transient event (e.g., removal of the user interface), a user transient event (e.g., moving from a warm room to a cooler room), a respiratory therapy system transient event (e.g., turning the respiratory therapy device on or off), or another transient event. For example, detection of a longer color rebound time at certain locations with respect to the face mapping after removal of the user interface may indicate a good fit.

Another example characteristic is a localized contour on the face of the user. This characteristic can be a detected contour on the face of the user, which can be compared with previously detected contours at that same location (e.g., a previous time the user was wearing the user interface) or current contours detected at adjacent locations. For example, if an indentation of a certain amount is detected on the user's skin around the entire seal of a user interface, it may be indicative of a strong seal and good fit.

Another example characteristic is a localized contour rebound on the face of the user. A localized contour rebound can include a change in contour over a period of time at a certain location (e.g., a certain location on the face mapping). The localized contour rebound may follow a transient event, such as a user interface transient event (e.g., removal of the user interface), a user transient event (e.g., moving from a warm room to a cooler room), a respiratory therapy system transient event (e.g., turning the respiratory therapy device on or off), or another transient event. For example, detection of a longer contour rebound time at certain locations with respect to the face mapping after removal of the user interface may indicate a good fit.

Another example characteristic is a localized contour on the user interface. This characteristic can be a detected contour on the user interface (e.g., on a part of the user interface, such as the seal of the user interface), which can be compared with previously detected contours at that same location (e.g., a previous time the user interface was worn) or current contours detected at other locations on the user interface. For example, if a certain amount of indentation is detected on the seal of the user interface, it may be indicative of a strong seal and good fit.

Another example characteristic is a localized contour rebound on the user interface. A localized contour rebound can include a change in contour over a period of time at a certain location on the user interface (e.g., a certain location on the user interface mapping). The localized contour rebound may follow a transient event, such as a user interface transient event (e.g., removal of the user interface), a user transient event (e.g., moving from a warm room to a cooler room), a respiratory therapy system transient event (e.g., turning the respiratory therapy device on or off), or another transient event. For example, detection of a longer contour rebound time at certain locations on the seal of the user interface may be indicative of a poor fit and/or a failing seal (e.g., a failing seal may not rebound as quickly).

Another example characteristic is a localized temperature on the user interface. This characteristic can be a detected temperature at some location on the user interface (e.g., on a part of the user interface, such as the seal), which can be compared with previously detected temperatures at that same location (e.g., a previous time the user interface was worn) or current temperatures detected at other locations on the user interface. For example, if a cold spot is detected at a location on the seal of a user interface, but other portions of the user interface are detected as being warmer, that cold spot may be indicative of an unintentional air leak. In another example, the length of time it takes for a portion of the user interface or the conduit to heat up to a given temperature may be indicative of a good or poor fit.

Another example characteristic is a vertical position of the user interface with respect to the one or more features of the face of the user. The vertical position of the user interface with respect to the one or more features of the face of the user can be based on the face mapping and sensor data and/or a user interface mapping. In an example, if the user interface, or a detected feature of the user interface, is detected to be too high or too low with respect to one or more features of the user's face (e.g., chin, mouth, and/or nose), it may be indicative of a poor fit (e.g., the straps may be pulling the user interface too high or too low).

Another example characteristic is a horizontal position of the user interface with respect to the one or more features of the face of the user. The horizontal position of the user interface with respect to the one or more features of the face of the user can be based on the face mapping and sensor data and/or a user interface mapping. In an example, if the user interface, or a detected feature of the user interface, is detected to be too far to one side of the user's face or another, it may be indicative of a poor fit (e.g., the straps may be pulling the user interface too much to the user's right or left side).

Another example characteristic is a rotational orientation of the user interface with respect to the one or more features of the face of the user. Rotational orientation can be a measure of how far the user interface is rotated, with respect to the user's face, about an axis of rotation extending out from the user's face, such as an axis of rotation extending from the face in an anterior or ventral direction. The rotational orientation of the user interface with respect to the one or more features of the face of the user can be based on the face mapping and sensor data and/or a user interface mapping. In an example, if the user interface, or a detected feature of the user interface, is detected to be rotated with respect to one or more features of the user's face by too high a degree, it may be indicative of a poor fit (e.g., the straps may be twisting the user interface on the user's face).

Another example characteristic is a distance between an identified feature of the user interface with respect to the one or more features of the face of the user. This distance can be based on the face mapping and sensor data and/or a user interface mapping. In an example, if a detected feature of the user interface (e.g., a vent), is detected to be too far displaced from a detected feature of the user face (e.g., the nose bridge), it may be indicative of a poor fit. This distance can be measured in one, two, or three dimensions. In an example, a user interface that is fit too loosely on a user's face may have a vent that is positioned a relatively large distance from the surface of the user's face, which may be indicative of a poor fit. In such a case, a measurement of a distance between the vent and a feature of the user's face may indicate the poor fit, which might be corrected by tightening of straps of the user interface.

In another example, identifying a leak characteristic at block 1514 can include determining a breathing pattern of the user based on received sensor data from block 1502, determining a thermal pattern associated with the face of the user based on received sensor data from block 1502 and the face mapping from block 1510, then using the breathing pattern and thermal pattern to generate a leak characteristic signal that is indicative of a balance between intentional vent leak and unintentional seal leak. For example, using the breathing pattern and the thermal pattern, an intentional vent leak signal can be generated indicating instances of, and optionally intensity of, intentional vent leak. A similar unintentional seal leak signal can be generated for unintentional seal leak. The leak characteristic signal can be a ratio between the unintentional seal leak signal and the intentional seal leak signal.

In some cases, identifying a characteristic at block 1514 can include identifying and confirming a potential characteristic at block 1516. Identifying and confirming a potential characteristic can include using a first set of sensor data to identify the characteristic, then using a second set of sensor data to confirm the characteristic. The first set of sensor data and the second set of sensor data can be collected from the same sensor(s) but at different times, or can be collected from different sensors. For example, collecting from the same sensor at different times can include identifying a potential unintentional air leak from thermal data while the user is wearing the user interface (e.g., detecting a localized change in surface temperature of the user's face across a region around the perimeter of the user interface), then confirming that unintentional air leak using thermal data acquired after the user has removed the user interface (e.g., detecting a lack of change in surface temperature of the user's face at a particular region over a duration of time, such as while blood rushes back into previously compressed tissue at other locations, but not adjacent the potential air leak).

In another example, collecting sensor data from different sensors to identify and confirm a potential characteristic can include using the thermal data collected while the user is wearing the user interface to identify the potential unintentional air leak, then using audio data collected at the same time to confirm the unintentional air leak (e.g., by detecting a characteristic audio or acoustic signal indicative of an unintentional air leak that coincides with the same time the potential unintentional air leek is detected in thermal image).

In some cases, additional sensor data can be received from a respiratory device at block 1520 and used in the identification of a characteristic at block 1514. Additional sensor data from a respiratory therapy device can include data acquired from a respiratory therapy system, such as respiratory therapy system in FIG. 1. Such additional sensor data can include data such as flow rate, pressure, delivered air temperature, ambient temperature, delivered air humidity, ambient humidity, ambient audio signals, audio signals conveyed via the conduit or air within the conduit, and the like. In some cases, additional sensor data can include data collected by sensors in the user interface and/or the conduit. In some cases, the additional sensor data from block 1520 is associated with the delivery of air by the respiratory therapy system. For example, additional sensor data associated with a delivered air temperature can be used at block 1514 to identify and/or confirm an unintentional air leak by comparing that delivered air temperature with temperatures of the user's skin surrounding a seal of the user interface.

In some cases, however, additional sensor data from block 1520 can be associated with a current fit of the user interface. In an example, the respiratory therapy system may include one or more sensors capable of detecting information pertaining to the user's face or the user interface. For example, an RF sensor in a respiratory therapy device may be able to detect ranging information associated with the current fit of the user interface on the user. In another example, one or more optical sensors (e.g., a visible light camera, passive infrared sensor, and/or active infrared sensor) may be used to detect information pertaining to the user's face and/or the user interface.

In some cases, historical data can be received at block 1522 and used in the identification of a characteristic at block 1514. Historical data can include historical sensor data (e.g., sensor data collected or received at previous instances of block 1502), historical characteristic data (e.g., information associated with characteristics identified at previous instances of block 1514), and/or other historical data (e.g., previous fit scores). At block 1518, identifying a characteristic at block 1514 can include comparing historical data received at block 1522 with sensor data received at block 1502. For example, receiving historical data at block 1522 can include accessing a memory containing a thermal mapping of the user's face prior to wearing a user interface. In such an example, at block 1518, that thermal mapping can be compared with a current thermal mapping of the user's face, which may have been taken while the user is wearing the user interface and/or after the use has removed the user interface. The detected differences in the two thermal mappings can be used to identify a characteristic (e.g., an unexpected localized temperature and/or an unintentional air leak).

In another example, receiving historical data at block 1522 can include accessing a memory containing previous contour rebound times associated with a particular user interface's seal. In such an example, the previous contour rebound times can be compared with newly acquired contour rebound times for that same portion of the user interface's seal. This comparison can indicate a degradation of the seal over time, which may cause poor fit and which might inevitably lead to seal failure. Therefore, this degradation can be detected as a characteristic and used to notify the user to replace the seal.

In some cases, identifying a characteristic can be facilitated or implemented using a machine-learning algorithm. Such a machine-learning algorithm can be trained using a training set of sensor data that has been collected by one or more users wearing a user interface or otherwise engaging in user interface transient events. The training data can include information about characteristics that were determined to exist, although that need not always be the case. In some cases, the training data can include information about the quality of fit of the user interface(s). Such quality of fit information can be based on subjective evaluation of the users, objective values collected with the use of other equipment (e.g., laboratory sensors and equipment, such as user interfaces outfit with specialized sensors and/or specialized sensing equipment), and the like. In some cases, characteristics identified at block 1514 are features used by the machine-learning algorithm.

In some cases, identifying characteristic(s) associated with the current fit can include determining a predicted quality of fit for a given user's face (e.g., face mapping from block 1510) and a given user interface (e.g., user interface mapping from block 1512 or other user interface identification information). In such cases, a face mapping of the user's face can be applied to design parameters for the given user interface to determine a predicted quality of fit (e.g., the best possible fit for that particular user interface on that user's face). If that predicted quality of fit is below a threshold, a determination can be made that the given user interface is not suitable for use by the user. The predicted quality of fit can further be used to determine whether a given evaluation of current fit (e.g., evaluation as described with reference to block 1528) can be improved upon (e.g., whether or not an improvement in current fit is achievable or likely). For example, if the predicted quality of fit for a given user interface on a particular user's face is “good” (e.g., out of “poor,” “ok,” “good,” and “very good,” although other measures for representing quality of fit can be used) and the current fit is evaluated as “good,” it can be determined that no further improvement on quality of fit is likely without changing user interfaces. In another example, if the predicted quality of fit is “very good” and the current fit is evaluated as “ok,” it can be determined that adjustment of other factors (e.g., other than changing the user interface) may improve the current fit, such as trimming facial hair, removing cosmetics, changing position in bed, or other changes.

At block 1524, output feedback can be generated based on the identified characteristic(s) from block 1514 and optionally the characteristic location (e.g., location with respect to the face mapping and/or user interface mapping). Generating output feedback at block 1524 can include presenting the output feedback, such as via a GUI, a speaker, a haptic feedback device, or the like. In some cases, output feedback can be presented as overlays on an image of the user, such as in the form of an augmented reality overlay. For example, icons, highlighting, text, and other indicia can be overlaid on an image (e.g., live or non-live) of the user to provide feedback regarding the quality of the current fit and/or instructions for how to improve the current fit.

In some cases, output feedback can include one or more characteristics that have been identified and optionally location information. For example, output feedback can be an image or representation of the user's face (e.g., taken from a visual spectrum camera, a thermal imager, or graphically generated from the face mapping) that is overlaid with graphics or text indicating the presence of detected characteristics. In an example, a detected unintentional air leak can be indicated over the image or representation of the user's face by an arrow, highlighted circle, or other attention-provoking visual element, at the detected location of the unintentional air leak.

In some cases, generating output feedback at block 1524 can include determining and presenting a suggested action to improve fit at block 1526. Determining and presenting a suggested action to improve fit can include using an identified characteristic and its location information to select an action designed to improve fit, then presenting that action (e.g., via text, graphic, audio feedback, haptic feedback, and the like). In some cases, the action to improve fit can be identified based on a lookup table or algorithm that can select the action to be taken based on the identified characteristic, optionally including its location information. For example, detection of an unintentional air leak may be correlated with the suggested action of adjusting a strap of the user interface, in which case the location of the unintentional air leak can be used to determine which strap is to be adjusted.

In some cases, however, determining an action to be taken can be based on a machine-learning algorithm. Such a machine-learning algorithm can be trained using a training set of actions to be taken and quality of fit information before and/or after the actions have been taken. Such quality of fit information can be based on subjective evaluation of the users, objective values collected with the use of other equipment (e.g., laboratory sensors and equipment, such as user interfaces outfit with specialized sensors and/or specialized sensing equipment), and the like. In some cases, characteristics identified at block 1514 are features used by this machine-learning algorithm.

In some cases, generating output feedback at block 1524 can include generating an evaluation of the current fit at block 1526. Generating an evaluation of the current fit at block 1526 can include using sensor data, identified characteristic(s), and/or characteristic location information. The evaluation can be a numerical score (e.g., a fit score, such as a numerical score between 0 and 100), a categorical score (e.g., textual scores, such as “good” “fair” and “poor”; color scores, such as green, yellow, and red; or graphical scores, such as a seal depicting no air leak, a seal depicting a small air leak, and a seal depicting a large air leak).

In some cases, generating the evaluation can include generating the evaluation based on an equation algorithm using as inputs sensor data, identified characteristic(s), and/or characteristic location information. For example, a fit score can be a weighted calculation of values associated with different identified characteristics (e.g., amount of temperature difference, distance of temperature difference from detected edge of user interface seal, duration of detected unintentional leak in audio signal, and the like).

In some cases, generating the evaluation can be based, at least in part, on comparisons with historical data (e.g., historical data received at block 1522). In such cases, the evaluation can be based, at least in part, on whether values associated with identified characteristic(s) are shown to be improving or degrading, in which case an improvement may warrant an increase in a fit score from a previous fit score and a degradation may warrant a decrease in a fit score from a previous fit score.

In some cases, generating the evaluation can be based on a machine-learning algorithm. Such a machine-learning algorithm can be trained using a training set of sensor data, identified characteristic(s), and/or characteristic location information. In some cases, the training set can include quality of fit information. Such quality of fit information can be based on subjective evaluation of the users, objective values collected with the use of other equipment (e.g., laboratory sensors and equipment, such as user interfaces outfit with specialized sensors and/or specialized sensing equipment), and the like. In some cases, characteristics identified at block 1514 are features used by this machine-learning algorithm.

While the process 1500 has been shown and described herein as occurring in a certain order, more generally, the various blocks of the process 1500 can be performed in any suitable order, and with fewer and/or additional blocks. For example, in some cases, blocks 1520, 1522, 1512, and 1518 are not used.

The flow diagrams in FIGS. 9-10 and 14-15 are representative of example machine readable instructions for collecting and analyzing data to select an optimal interface for respiratory pressure therapy and conduct follow up analysis such as fit of the selected user interface. In this example, the machine readable instructions comprise an algorithm for execution by: (a) a processor; (b) a controller; and/or (c) one or more other suitable processing device(s). The algorithm may be embodied in software stored on tangible media such as flash memory, CD-ROM, floppy disk, hard drive, digital video (versatile) disk (DVD), or other memory devices. However, persons of ordinary skill in the art will readily appreciate that the entire algorithm and/or parts thereof can alternatively be executed by a device other than a processor and/or embodied in firmware or dedicated hardware in a well-known manner (e.g., it may be implemented by an application specific integrated circuit [ASIC], a programmable logic device [PLD], a field programmable logic device [FPLD], a field programmable gate array [FPGA], discrete logic, etc.). For example, any or all of the components of the interfaces can be implemented by software, hardware, and/or firmware. Also, some or all of the machine readable instructions represented by the flowcharts may be implemented manually. Further, although the example algorithms are described with reference to the flowcharts illustrated in FIGS. 9-10 and 14-15, persons of ordinary skill in the art will readily appreciate that many other methods of implementing the example machine readable instructions may alternatively be used. For example, the order of execution of the blocks may be changed, and/or some of the blocks described may be changed, eliminated, or combined.

As used in this application, the terms “component,” “module,” “system,” or the like, generally refer to a computer-related entity, either hardware (e.g., a circuit), a combination of hardware and software, software, or an entity related to an operational machine with one or more specific functionalities. For example, a component may be, but is not limited to being, a process running on a processor (e.g., digital signal processor), a processor, an object, an executable, a thread of execution, a program, and/or a computer. By way of illustration, both an application running on a controller, as well as the controller, can be a component. One or more components may reside within a process and/or thread of execution, and a component may be localized on one computer and/or distributed between two or more computers. Further, a “device” can come in the form of specially designed hardware; generalized hardware made specialized by the execution of software thereon that enables the hardware to perform specific function; software stored on a computer-readable medium; or a combination thereof.

The terminology used herein is for the purpose of describing particular embodiments only, and is not intended to be limiting of the invention. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art. Furthermore, terms, such as those defined in commonly used dictionaries, should be interpreted as having a meaning that is consistent with their meaning in the context of the relevant art, and will not be interpreted in an idealized or overly formal sense unless expressly so defined herein.

One or more elements or aspects or steps, or any portion(s) thereof, from one or more of any of claims 1 to 100 below can be combined with one or more elements or aspects or steps, or any portion(s) thereof, from one or more of any of the other claims 1 to 100 or combinations thereof, to form one or more additional implementations and/or claims of the present disclosure.

While various embodiments of the present invention have been described above, it should be understood that they have been presented by way of example only, and not limitation. Although the invention has been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application. Thus, the breadth and scope of the present invention should not be limited by any of the above described embodiments. Rather, the scope of the invention should be defined in accordance with the following claims and their equivalents.

1. A system for selecting an interface suited to a user's face for respiratory therapy, the system comprising:
a storage device for storing a facial image of the user;
a facial profile engine operable to determine facial features of the user based on the facial image;
one or more databases for storing:
a plurality of facial features from a user population and a corresponding plurality of interfaces used by the user population; and
operational data of respiratory therapy devices used by the user population with the plurality of corresponding interfaces; and
a selection engine coupled to the one or more databases, wherein the selection engine is operable to select an interface for the user from the plurality of corresponding interfaces based on a desired outcome from the stored operational data and the determined facial features.

2. (canceled)

3. The system of claim 1, wherein the interface is a mask and the respiratory therapy device is configured to provide one or more of a Positive Airway Pressure (PAP) or a Non-invasive ventilation (NIV).

4. The system of claim 1, wherein at least one of the respiratory therapy devices includes an audio sensor to collect audio data during the operation of the at least one of the respiratory therapy devices and wherein the selection engine is operable to analyze the audio data to determine a type of the corresponding interface of the at least one of the respiratory therapy devices based on matching the audio data to an acoustic signature of a known interface.

5. (canceled)

6. The system of claim 1, wherein the selection engine selects the interface based on demographic data of the user compared with demographic data of a population of users stored in the one or more databases.

7. (canceled)

8. The system of claim 1, wherein the facial image is determined from a scan from a mobile device including a camera, wherein the mobile device includes a depth sensor, and wherein the camera is a 3D camera, and wherein the facial features are three-dimensional features derived from a meshed surface derived from the facial image.

9-13. (canceled)

14. The system of claim 1, wherein the desired outcome is one of a seal between the interface and a facial surface to prevent leaks; or compliance with using the respiratory therapy device.

15. (canceled)

16. The system of claim 1, further comprising a mobile device operable to collect subjective data input from a user, and wherein the selection of the interface is based partly on the subjective data.

17. The system of claim 1, further comprising a machine learning module operable to determine types of operational data correlated with interfaces achieving the desired outcome.

18. (canceled)

19. The system of claim 1, wherein the selection engine is further operable to receive feedback from the user based on operating the selected interface and based on an undesirable result, select another interface based on the desired outcome, wherein the undesirable result is one of low user compliance with therapy, a high leak, or an unsatisfactory subjective result data.

20. (canceled)

21. A method to select a suitable interface to the face of a user for respiratory therapy, the method comprising:
storing a facial image of the user in a storage device;
determining facial features based on the facial images;
storing a plurality of facial features from a user population and a corresponding plurality of interfaces in one or more databases;
storing operational data of respiratory therapy devices used by a user population with the plurality of corresponding interfaces in one or more databases; and
selecting an interface for the user from the plurality of corresponding interfaces based on a desired outcome from the stored operational data and the determined facial features.

22-66. (canceled)

67. A method comprising:
receiving sensor data associated with a current fit of an interface on a face of a user, the sensor data collected by one or more sensors of a mobile device;
generating a face mapping using the sensor data, wherein the face mapping is indicative of one or more features of the face of the user;
identifying, using the sensor data and the face mapping, a first characteristic associated with the current fit, wherein the first characteristic is indicative of a quality of the current fit, and wherein the first characteristic is associated with a characteristic location on the face mapping;
generating output feedback based on the identified first characteristic and the characteristic location.

68. (canceled)

69. The method of claim 67, wherein generating the output feedback comprises:
determining a suggested action, which if implemented, would affect the first characteristic to improve the current fit; and
presenting the suggested action using an electronic interface of the mobile device.

70. The method of claim 67, wherein the sensor data includes infrared data from i) a passive thermal sensor; ii) an active thermal sensor; or iii) both i and ii; and wherein the sensor data includes distance data, wherein the distance data is indicative of one or more distances between the one or more sensors of the mobile device and the face of the user.

71-74. (canceled)

75. The method of claim 67, wherein the first characteristic includes:
i) a localized temperature on the face of the user;
ii) a localized change in temperature on the face of the user;
iii) a localized color on the face of the user;
iv) a localized change in color on the face of the user;
v) a localized contour on the face of the user;
vi) a localized change in contour on the face of the user;
vii) a localized contour on the interface;
viii) a localized change on the interface;
ix) a localized temperature on the interface; or
x) any combination of i through ix; and
wherein the characteristic location includes:
i) a vertical position of the interface with respect to the one or more features of the face of the user;
ii) a horizontal position of the interface with respect to the one or more features of the face of the user;
iii) a rotational orientation of the interface with respect to the one or more features of the face of the user;
iv) a distance between an identified feature of the interface with respect to the one or more features of the face of the user; or
v) any combination of i through iv.

76-78. (canceled)

79. The method of claim 67, further comprising generating an interface mapping using the sensor data, wherein the interface mapping is indicative of a relative position of one or more features of the interface with respect to the face mapping, and wherein identifying the first characteristic includes using the interface mapping.

80. (canceled)

81. The method of claim 67, further comprising:
identifying, using the sensor data and the face mapping, a second characteristic, the second characteristic being associated with a possible future failure of the interface; and
generating output feedback based on the identified second characteristic, the output feedback being usable to reduce a likelihood that the possible future failure will occur or to delay occurrence of the possible future failure.

82. The method of claim 67, further comprising accessing historical sensor data associated with one or more historical fits of the interface on the face of the user prior to receiving the sensor data, wherein identifying the first characteristic further uses the historical sensor data.

83. The method of claim 67, further comprising generating a current fit score using the sensor data and the face mapping, wherein the output feedback is generated to improve a subsequent fit score.

84-90. (canceled)

91. The method of claim 67, wherein the output feedback is usable to improve the current fit, the method further comprising:
generating an initial score based on the current fit using the sensor data;
receiving subsequent sensor data associated with a subsequent fit of the interface on the face of the user, wherein the subsequent fit is based on the current fit after implementation of the output feedback;
generating a subsequent score based on the subsequent fit using the subsequent sensor data; and
evaluating the subsequent score, wherein the subsequent score is indicative of an improvement in quality over the initial score.

92. The method of claim 67, wherein identifying the first characteristic associated with the current fit includes:
determining a breathing pattern of the user based on the received sensor data;
determining a thermal pattern associated with the face of the user based on the received sensor data and the face mapping; and
determining a leak characteristic using the breathing pattern and the thermal pattern, wherein the leak characteristic is indicative of a balance between intentional vent leak and unintentional seal leak.

93-100. (canceled)
